(19) United States
(12) Patent Application Publication
(10) Pub. No.: US 2025/0367084 A1
(43) Pub. Date: Dec. 4, 2025

(54) Micellar Composition

(71) Applicant: William Buchanan, Midhurst (GB)

(72) Inventor: William Buchanan, Midhurst (GB)

(73) Assignee: Phytoceutical Limited, Midhurst (GB)

(21) Appl. No.: 18/854,397

(22) PCT Filed: Dec. 19, 2023
(86) PCT No.: PCT/GB2023/053302

§ 371 Date: Oct. 4, 2024

(30) Foreign Application Priority Data
Dec. 23, 2022 (GB) 2219724.8

(51) Int. Cl. A61K 8/02; A61K 8/34; A61K 8/35; A61K 8/43; A61K 8/49; A61K 8/67; A61K 8/73; A61Q 19/00

(52) U.S. Cl. CPC A61K 8/0291; A61K 8/34; A61K 8/347; A61K 8/35; A61K 8/43; A61K 8/498; A61K 8/671; A61K 8/73; A61Q 19/00; A61K 2800/413; A61K 2800/48; A61K 2800/524

(57) ABSTRACT

A composition comprising micelles, the micelles comprising an aqueous outer phase that encapsulates an inner phase, wherein the micelles have a diameter of 100 nm or less, and wherein the outer phase comprises a compound of Formula (I). Further disclosures include cosmetic and/or therapeutic uses of the composition, and methods of preparing the composition.

### FIELD OF THE INVENTION

The present invention relates to a micellar composition, and to medical and/or cosmetic uses thereof. The micellar composition may be employed in the delivery of an active ingredient to the skin.

### BACKGROUND TO THE INVENTION

A micelle is a nanosized, colloidal carrier with a hydrophilic exterior shell and a hydrophobic interior core. The hydrophobic core provides a pocket in which poorly water-soluble components can be dissolved while the hydrophilic shell allows the micelles to remain stably dispersed in aqueous media.

Tocopheryl polyethylene glycol succinate (TPGS) is a water-soluble derivative of vitamin E that acts as a surfactant with the ability to form micellar nanoparticles in water. The TPGS molecule is amphiphilic, with a lipophilic alkyl tail (tocopherol succinate moiety) and a hydrophilic polar head (polyethylene glycol chain).

The stability of compounds such as retinoids (e.g. retinol), cannabidiol (CBD) and curcumin have been found to have poor absorbability through the human skin.

In addition, such compounds are affected by oxidation. In some cases, the oxidation of these compounds may be beneficial, as it can convert biologically inactive molecules into the active forms.

GB 2550346 and WO 2017/194965 describe a composition comprising micelles of aqueous d-α-tocopheryl polyethylene glycol 1000 succinate (TPGS) and retinol, wherein the micelles do not exceed 100 nm. The composition can be obtained by a method comprising the steps of a. dissolving retinol in an organic solvent to provide a hydrophobic phase; b. adding the hydrophobic phase into aqueous TPGS; and c. removing at least a portion of the organic solvent.

US 2021/0353589 discloses water-soluble cannabinoid formulations.

AU 20200394709 discloses pharmaceutical cannabinoid nano-micelle preparations.

WO 2020/002917 discloses formulations of peroxisome proliferator activated receptor (PPAR) modulators, such as curcumin, for the treatment or prevention of neurodegenerative conditions, disorders of the retina, and brain disorders, as well as pulmonary arterial hypertension, cancer and antifibrotic disorders.

US 2021/401746 discloses stabilised compositions comprising cannabinoids, for use as pharmaceuticals or nutraceutical products.

### SUMMARY OF THE INVENTION

According to a first aspect the present invention provides a composition comprising micelles (such that the composition is a micellar composition), the micelles comprising an aqueous outer phase that encapsulates an inner phase, wherein the micelles have a diameter of 100 nm or less (e.g. from 5 to 50 nm). The aqueous outer phase comprises a compound of Formula (I), for example tocopheryl polyethylene glycol succinate (TPGS), or a composition thereof.

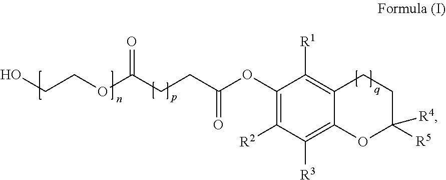

R¹, R², R³ and R⁴ are each independently H or a C1-6 hydrocarbon group that is optionally substituted by one or more Y group; R⁵ is a C6-30 hydrocarbon group; n is from 3 to 100; p is from 0 to 6; q is from 0 to 3; and Y is selected from the list consisting of ether, ester, sulfone, sulfoxide, amide, amine e.g. secondary amine and tertiary amine), boronate ester, ketone and aldehyde. The inner phase may be hydrophobic.

The composition of the first aspect may find (e.g. cosmetic and/or therapeutic) use to administer a biologically active compound (e.g. retinoids such as retinol, curcumin and/or cannabidiol) into the skin (e.g. the epidermis and/or dermis) and/or a mucous membrane. In some embodiments the biologically active compound may be curcumin and/or cannabidiol. The composition (e.g. when combined with a biologically active compound) may be used to reduce the appearance of wrinkles, reduce melanin production, lighten scar tissue, provide a more even skin tone, increase the radiance of skin, increase the thickness of skin, increase the elasticity of skin, reduce inflammation of skin, reduce and/or prevent blocked pores, and/or increase the plumpness of skin. The composition may find uses in reducing the oiliness of skin (e.g. reducing the excretion of sebum from the skin), reducing acne (e.g. by reducing the secretion or activation of inflammatory proteins), reduce (the appearance of) wrinkles, and/or reducing dryness or itchiness of skin (e.g. treating or preventing prurigo, lichen simplex and/or pruritus). The composition may therefore be used to reduce the (appearance of) skin ageing, minimise irritation and/or to provide additional benefits in terms of healthy skin ageing.

The composition may be for use as a medicament. The disease treated may depend upon the bio-active agent incorporated into the composition.

The present inventors have found that the micelles of the compositions of present invention provide particular benefits in relation to compositions for topical administration or application, for instance in relation to scar recovery and/or wound (e.g. pressure sore) care. The composition may be used to treat a disease that may be skin-related, such as acne, a wound, pemphigus, alopecia areata, psoriasis, dermatitis (e.g. atopic), epidermolysis bullosa, bacterial infection, rosacea, hidradenitis suppurativa, scleroderma and/or ichthyosis.

According to a second aspect the present invention provides a composition for use in a method of treatment, the method comprising topical administration of the composition, wherein the composition is as defined by the first aspect. The method may be therapeutic and/or not cosmetic.

According to a third aspect the present invention provides a method of cosmetic treatment comprising topical application of a composition to a subject's skin, wherein the composition is as defined by the first aspect. For example, the method may include applying the composition of the first aspect to the skin of a subject. The composition may be applied daily, for example for one week or more. Therefore, the composition may be a cosmetic composition. The composition may be for topical administration (particularly to the skin, especially the epidermis).

The present inventors have determined that the micelles are highly stable. For instance, it has also been shown that the micelles are themselves particularly stable over long periods of time (e.g. 150 days).

The micelles have been found to improve the stability of compounds encapsulated by the micelles. For example, the chemical stability (e.g. against oxidation) can be improved for compounds that are sensitive to oxidation (e.g. retinoids, curcumin and/or cannabidiol).

In particular, it has been shown that the micelles can slow the oxidative conversion of retinoids such as retinol to retinal. Retinal is subsequently oxidised to retinoic acid, which is the active compound for triggering the expression of fibroblasts and begin tissue generation. However, large amounts of retinoic acid can be toxic and/or cause irritation.

Thus, the inventors have determined that the micelles of the invention can be used to control the conversion of retinoids such as retinol to retinal, and therefore control the release of the active retinoic acid for subsequent use (e.g. in cosmetic applications). As such, the composition may comprise a biologically active compound (e.g. a retinoid such as retinol, curcumin and/or cannabidiol). The biologically active compound (or agent) may be present within the micelles). The composition may be a modified or controlled (e.g. slow) release composition. It will be understood that such compositions differ from immediate release compositions, where all of the active agent is available substantially immediately following administration.

Certain compounds, such as retinol, can irritate the skin, especially when applied in relatively high doses. The ability to control the release and/or oxidation of retinol can therefore prevent adverse skin reactions, such as allergic reactions and/or sensitivity in a subject, following application of the composition, for example compared to a composition that does not include the micelles and/or the compound of Formula (I), such as TPGS.

The compositions may therefore find particular use in relation to subjects (e.g. humans) that have sensitive skin (e.g. cutaneous sensory syndrome), for example where the subject has one or more symptoms such as itching, burning, stinging, allodynia, numbness, hypoaesthesia, irritation, erythema, and/or dryness. Skin sensitivities are more prevalent, and thus the compositions may find applications, in subjects that have atopic dermatitis, psoriasis, acne, rosacea, and/or seborrheic dermatitis, and/or conditions such as hyperalgesia, autoimmune connective tissue diseases (e.g. dermatomyositis, systemic lupus erythematosus, Sjogren syndrome, systemic sclerosis, and/or mixed connective tissue disease) insulin resistance (e.g. diabetes) and/or hyperglycemia. The sensitivity may be caused by hypersensitivity, for example to a medication and/or skin care product. In particular, the compositions may find application for a subject that has sensitive skin, or a condition that may increase the prevalence of sensitive skin, and another skin-related condition, such as acne, a wound, pemphigus, alopecia areata, psoriasis, dermatitis (e.g. atopic), epidermolysis bullosa, bacterial infection, rosacea, hidradenitis suppurativa, scleroderma and/or ichthyosis.

Certain compounds, such as retinol, can increase the sensitivity of skin to sun exposure. Therefore, the compositions of the invention, which may induce a lower level of sensitivity due to their controlled release nature, may be safely applied (e.g. without a sun protection product) before sun exposure (e.g. in the morning), when traditional compositions would not be safely applied at such times (e.g. without a sun protection product).

Owing to the controlled release nature of the compositions, the compositions may be applied or administered at a lower frequency compared to traditional compositions.

For instance, the composition may be applied or administered at a frequency of once a day or less, such as once every two days, or once every three days, or once every four or five days, such as once a week. The frequency may be from once every day to once every month, such as from once every day to once every two weeks, for example from once every two days to once every month.

The stability provided by the micelles has been shown to be particularly good when the composition comprises a compound such as PHMB and/or xanthan gum, for example in the outer (aqueous) phase of the composition. Other thickeners and/or stabilisers are also thought to be effective.

The claimed compositions have been found to provide excellent transfer of active compounds (e.g. retinoids such as retinol, curcumin and/or cannabidiol) to the skin, and minimal transfer of the active compounds through the skin. Therefore, the compositions allow the active compounds to be particularly effective within the skin whilst simultaneously preventing significant entry of the active compounds into the bloodstream and, as such, reducing or preventing any toxic effects of the active compounds.

The use may achieve a controlled release of the biologically active compound. The use may achieve low transfer of the biologically active compound through the skin (i.e. passing all of the way through the skin, e.g. through the dermis).

In particular, the inventors have shown that retinoids such as retinol are cytotoxic to fibroblasts at levels of 10 μg/mL and above. Therefore, controlling the release of retinoids such as retinol is important to ensure that the benefits of these compounds can be achieved with minimal side effects.

By controlling the dosage and release of retinoids, fibroblast proliferation and collagen production can be controlled to optimise healthy tissue generation.

The interplay between fibrotic tissue, such as scar formation, and collagen formation, which is non-fibrotic, is important. It has recently been determined that fibrotic tissue replicates in a similar way to non-fibrotic tissue. Keratinocytes and fibroblasts communicate with each other via double paracrine signalling loops, known as cross talk or dynamic reciprocity, which coordinate their actions to restore normal tissue homeostasis after wounding. So, by controlling dose and release intervention, fibroblasts can be influenced to generate non-fibrotic tissue.

The use may achieve a level of the biologically active compound in the skin of 1 wt % or more (relative to the amount of compound applied), such as 2 wt % or more or 2.5 wt % or more, or 3 wt % or more, and/or achieve a level of the biologically active compound transferred through the skin of less than 1 wt % (relative to the amount of the biologically active compound applied), such as 0.8 wt % or less, or 0.5 wt % or less.

The use may reduce irritation of the skin, for example compared to the irritation observed when the biologically active compound is administered to the skin in a solution of the same concentration and in the same solvent but that does not contain micelles.

The composition may remain suitable for use following a period of 100 days or more, such as 150 days or more, Such as from 100 days to 700 days, or from 150 days to 400 days.

In a fourth aspect, the present invention provides a method of producing a micellar composition as defined in the first aspect, the method comprising: providing a hydrophobic phase comprising an organic solvent; and adding the hydrophobic phase into an aqueous dispersion (e.g. solution) of a compound of Formula (I) as defined in relation to the first aspect (e.g. TPGS); and optionally removing at least a portion of the organic solvent.

The stability of the micelles may be improved when a stabiliser and/or thickener (e.g. polyvinyl caprolactam-polyvinyl acetate-polyethylene glycol graft copolymer) is added to the hydrophobic phase prior to combination with the aqueous dispersion of the compound of Formula (I), e.g. TPGS. Thus, the hydrophobic phase comprising an organic solvent may also comprise a stabiliser and/or thickener (e.g. polyvinyl caprolactam-polyvinyl acetate-polyethylene glycol graft copolymer).

This method may differ from previous methods in that the hydrophobic (i.e. lipophilic) phase is added to the aqueous dispersion of the compound of Formula (I), e.g. TPGS, whereas previous related methods have added the aqueous dispersion of the compound of Formula (I), e.g. TPGS, to the hydrophobic phase, before diluting.

Preferably the hydrophobic phase does not include fatty acid esters or other hydrophobic hydrocarbons (e.g. acetyl palmitate and/or soy oil). Preferably the hydrophobic phase includes a retinoid (e.g. retinol), CBD and/or curcumin. In particular, the hydrophobic phase may include CBD and/or curcumin. It is surprising that (as shown by the Examples) micelles can be made including such compounds as difficulties have been experienced with other lipophilic materials, such as soy oil.

It will be understood that a disclosure in relation to one aspect of this invention may equally apply to another aspect, for example in relation to the cosmetic and medical uses of the compositions.

US 2021/0353589 and US 2021/401746 do not disclose the specific stabilisation of compositions using PHMB, or other specific features of the present invention.

AU 20200394709 and WO 2020/002917 do not disclose the use of PHMB or xanthan gum, or other specific features of the present invention.

The present disclosure includes the subject-matter of the following clauses:
1. A composition comprising micelles, the micelles comprising an aqueous tocopheryl polyethylene glycol succinate (TPGS) outer phase that encapsulates an inner phase, wherein the micelles have a diameter of 5 to 50 nm.
2. The composition of clause 1, wherein the micelles have a diameter of (i) 18 to 20 nm; (ii) 21 to 25 nm; or (ii) 22 to 24 nm.
3. The composition of clause 1 or clause 2, wherein the inner phase comprises retinol, cannabidiol, and/or curcumin.
4. The composition of clause 3, wherein the inner phase comprises retinol, such as 0.1 to 3w/w % retinol.
5. The composition of clause 3, wherein the inner phase comprises cannabidiol (CBD), such as (i) 0.01 to 2w/w % CBD; (ii) 0.1 to 1w/w % CBD; (iii) 0.08 to 0.12w/w % CBD; (iv) 0.8 to 1.2w/w % CBD or (v) 0.4 to 0.6w/w % CBD.
6. The composition of clause 3, wherein the inner phase comprises curcumin, such as (i) 0.1 to 1w/w % curcumin by volume; ii) 0.3 to 0.7w/w % curcumin; or (iii) 0.4 to 0.6 w/w % curcumin.
7. The composition of any one of the preceding clauses, wherein the aqueous TPGS outer phase additionally comprises polysorbate.
8. The composition of any one of the preceding clauses, additionally comprising a thickener and/or a stabiliser, optionally wherein the thickener and/or stabiliser is selected from one or more of xanthan gum; hyaluronic acid; polyhexamethylene biguanide (PHMB); hydroxy ethyl cellulose; collagen (including collagen peptides); clays (e.g. bentonite/hectorite clays); alginates; cellulose and esters thereof; gellan gums, polymethacrylates including glyceryl; acrylates; starches including potato; hydroxypropyl cellulose; methylcellulose; chitosan; polyvinyl alcohol; hydroxypropyl methylcellulose; carbomer; Alginate hydrofibre, films, foams, amorphous gels, hydrocolloids, hydrogels, non-adherent contact layers, and carboxymethylcellulose.
9. The composition of clause 8, wherein the thickener and/or stabiliser comprises (i) xanthan gum; (ii) hyaluronic acid; and/or (iii) polyhexamethylene biguanide (PHMB).
10. The composition of any one of the preceding clauses, wherein the TPGS comprises TPGS 200, TPGS 300, TPGS 400, TPGS 750, TPGS 1000, TPGS 1500, TPGS 2000, TPGS 3000 and/or TPGS 4000, preferably TPGS 1000.
11. A cosmetic treatment comprising applying the composition of any one of the preceding clauses to a subject's skin.
12. The cosmetic treatment of clause 12, wherein the composition is applied to the subject's skin in an amount of 0.025 mg (0.01% retinol) to 84 mg (5% retinol) per cm².
13. The cosmetic treatment of clause 11 or clause 12, wherein the composition is applied to provide 500 mg retinol per cm² of skin; and/or wherein the composition is applied daily for one week or more.
14. The composition of any one of clauses 1 to 10 for use as a medicament.
15. A method for the preparation of the composition of any one of clauses 1 to 10 comprising providing a hydrophobic phase comprising an organic solvent; adding the hydrophobic phase into aqueous TPGS; and optionally removing at least a portion of the organic solvent.
16. The method of clause 15, wherein the hydrophobic phase comprises ethanol and/or polysorbate.

### DETAILED DESCRIPTION

### Biologically Active Agents

The micelles containing the compound of Formula (I) (e.g. TPGS) may be used to encapsulate a range of components (e.g. bio-active agents) as cargo. Typically, the composition may comprise a retinoid, cannabidiol, and/or curcumin. Preferably the inner phase comprises retinaldehyde, retinol, cannabidiol, and/or curcumin. FIG. 16 shows that it is possible to include more than one bio-active agent in the composition. Retinoids include retinol, retinal, tretinoin (retinoic acid), isotretinoin, alitretinoin, etretinate, acitretin, adapalene, bexarotene, tazarotene, and trifarotene. Preferred retinoids include retinol and retinal.

Especially when combined with retinoids (e.g. retinol), the composition may be used to reduce (the appearance of) wrinkles, reduce melanin production, lighten scar tissue, provide a more even skin tone, increase the radiance of skin, increase the thickness of skin, increase the elasticity of skin, reduce inflammation of skin, reduce and/or prevent blocked pores, and/or increase the plumpness of skin.

Curcumin is the main active ingredient in turmeric, which has powerful antioxidant and anti-inflammatory properties. Curcumin helps reduce excess production of melanin which in turn lightens scars and keeps even the skin tone.

Especially when combined with curcumin, the composition may be used to reduce melanin production, lighten scar tissue and/or provide a more even skin tone.

Most clinical evidence relating to cannabinoids, such as CBD, to date has focused on the effects of CBD and other cannabinoids when consumed, inhaled, or injected. There is limited research investigating the therapeutic potential for topical applications.

Yet, there is evidence to suggest applying cannabinoids, and specifically CBD, topically may be used for the treatment of diseases such as acne, seborrhea, eczema/dermatitis, and skin barrier function. Topical administration or application of cannabinoids, such as CBD may be particularly effective in the treatment of disorders relating to the skin, in the improvement of skin health generally, and/or in the appearance of skin. This may be due to the human endocannabinoid system ECS playing an important regulatory function in the skin.

Although CBD has a reasonably high molecular weight (314.46 Da), its high log P value of ˜6.3, poses unique challenges to its transdermal delivery. However, this challenge may be overcome if appropriate carrier systems are used, as set out in the present invention.

CBD may find particular use in reducing the oiliness of skin (e.g. reducing the excretion of sebum from the skin), reducing acne (e.g. by reducing the secretion or activation of inflammatory proteins), reduce (the appearance of) wrinkles, and/or reducing dryness or itchiness of skin (e.g. treating or preventing prurigo, lichen simplex and/or pruritus).

The amount of bio-active agent (e.g. retinoid such as retinol, curcumin and/or cannabidiol) in the inner phase of the micelles and/or in the overall composition may be 0.00001 wt % or more, or 0.0001 wt % or more, such as 0.001 wt % or more, such as 0.01 wt % or more, or 0.05 wt % or more, preferably 0.08 wt % or more, or 0.1 wt % or more, such as 0.5 wt % or more, or 0.6 wt % or more, or 0.8 wt % or more, or 1 wt % or more. The amount may be 30 wt % or less, such as 20 wt % or less, preferably 10 wt % or less, for example 8 wt % or less, or 5 wt % or less, such as 4 wt % or less, or 3 wt % or less, or 2 wt % or less, for example 1.5 wt % or less, or 1.2 wt % or less, or 1 wt % or less, such as 0.6 wt % or less. The amount of the bio-active agent in the inner phase of the micelles and/or in the overall composition may be from 0.00001 wt % to 30 wt %, such as from 0.01 to 10 wt %, or from 0.08 to 5 wt %, or from 0.1 to 3 wt %.

The amount of retinoid such as retinol or retinal in the inner phase may be 0.00001 to 3w/w %. The amount of retinoid such as retinol or retinal in the inner phase may be 0.1 to 3w/w %. The amount of cannabidiol in the inner phase may be 0.00001 to 2 w/w %, such as 0.0001 to 1w/w % CBD; 0.0008 to 0.12w/w % CBD; 0.0008 to 1.2w/w % CBD; or 0.4 to 0.0006w/w % CBD. The amount of cannabidiol in the inner phase may be 0.01 to 2 w/w %, such as 0.1 to 1w/w % CBD; 0.08 to 0.12w/w % CBD; 0.8 to 1.2w/w % CBD; or 0.4 to 0.6w/w % CBD. The inner phase may comprise curcumin in an amount of 0.00001 to 1w/w %, such as 0.0003 to 0.7 w/w % curcumin, or 0.0004 to 0.6 w/w % curcumin. The inner phase may comprise curcumin in an amount of 0.1 to 1w/w %, such as 0.3 to 0.7 w/w % curcumin, or 0.4 to 0.6 w/w % curcumin.

The composition may include a biologically active agent (e.g. retinoid such as retinol, curcumin and/or cannabidiol) in an amount of 1 ng/mL or more, such as 10 ng/ml or more, or 100 ng/ml or more, for example 1 μg/mL or more, such as 10 μg/mL or more, or 20 μg/mL or more. The dose per cm² of skin may be 2000 μg/mL or less, or 1500 μg/mL or less, preferably 1000 μg/mL or less, such as 800 μg/mL or less, or 700 μg/mL or less, or 600 μg/mL or less. More preferably the dose of biologically active agent per cm² of skin is 400 μg/mL or less, such as 200 μg/mL or less, or 100 μg/mL or less, yet more preferably 75 μg/mL or less, such as 50 μg/mL or less, more preferably 30 μg/mL or less, or 20 μg/mL or less, especially 15 μg/mL or less, or 10 μg/mL or less, such as 8 μg/mL or less, or 5 μg/mL or less. The dose per cm² of skin may be from 1 ng/mL to 2000 μg/mL, such as from 10 ng/mL to 1000 μg/mL, or from ng/mL to 800 μg/mL. Preferably the dose per cm² of skin is from 1 ng/ml to 100 μg/mL, such as from 1 ng/mL to 50 μg/mL, or from 1 ng/mL to 15 μg/mL, such as from 1 ng/ml to 10 μg/mL.

Preferably, the bio-active (therapeutic) agent is an oil-soluble active agent. Preferably, the oil-soluble active agent is an oil-soluble vitamin, preferably an oil-soluble aromatic vitamin. For example the bio-active therapeutic agent may be tocotrienol and/or tocopherol. The bio-active agent may be other oil-soluble active agents, for example, retinyl palmitate.

### Thickeners and Stabilisers

The composition (e.g. the outer phase) may comprise a thickener and/or stabiliser, for example one or more of: xanthan gum; hyaluronic acid; polyhexamethylene biguanide (PHMB, which may be buffered); polyvinyl caprolactam-polyvinyl acetate-polyethylene glycol graft copolymer; hydroxy ethyl cellulose; collagen (including collagen peptides); clays (e.g. bentonite/hectorite clays); alginates; cellulose and esters thereof; gellan gums, polymethacrylates including glyceryl; acrylates; starches including potato; hydroxypropyl cellulose; methylcellulose; chitosan; polyvinyl alcohol; hydroxypropyl methylcellulose; carbomer; alginate hydrofibre, films, foams, amorphous gels, hydrocolloids, hydrogels, non-adherent contact layers, and carboxymethylcellulose.

Preferably the composition (e.g. the outer phase) comprises polyvinyl caprolactam-polyvinyl acetate-polyethylene glycol graft copolymer, polysorbate, xanthan gum and/or PHMB. The stabiliser may be polysorbate (e.g. polysorbate 40). The composition may include two or more stabiliser and/or thickeners, for example one compound selected from the list consisting of polyvinyl caprolactam-polyvinyl acetate-polyethylene glycol graft copolymer polysorbate, xanthan gum and PHMB, and another compound selected from the list consisting of xanthan gum; hyaluronic acid; polyhexamethylene biguanide (PHMB, which may be buffered); polyvinyl caprolactam-polyvinyl acetate-polyethylene glycol graft copolymer; hydroxy ethyl cellulose; collagen (including collagen peptides); clays (e.g. bentonite/hectorite clays); alginates; cellulose and esters thereof; gellan gums, polymethacrylates including glyceryl; acrylates; starches including potato; hydroxypropyl cellulose; methylcellulose; chitosan; polyvinyl alcohol; hydroxypropyl methylcellulose; carbomer; alginate hydrofibre, films, foams, amorphous gels, hydrocolloids, hydrogels, non-adherent contact layers, and carboxymethylcellulose.

Without being bound by theory, the PHMB may “wrap” around the micelles, and/or form “nests” or folded shapes depending on the buffers. This is indicated by a slight increase in micelle size following addition of PHMB.

The amount of thickener and/or stabiliser (e.g. PHMB and/or xanthan gum) in the composition or in the outer phase may be 0.00001 wt % or more, or 0.0001 wt % or more, such as 0.001 wt % or more, such as 0.01 wt % or more, or 0.05 wt % or more, or 0.08 wt % or more, such as 0.1 wt % or more, or 0.2 wt % or more, or 0.25 wt % or more, such as 0.28 wt % or more, or 0.3 wt % or more. The amount may be 30 wt % or less, such as 20 wt % or less, preferably 10 wt % or less, for example 8 wt % or less, or 5 wt % or less, such as 4 wt % or less, or 3 wt % or less, more preferably 2 wt % or less, for example 1.5 wt % or less, or 1.2 wt % or less, or 1 wt % or less, such as 0.6 wt % or less, such as 0.5 wt % or less, or 0.4 wt % or less, or 0.3 wt % or less. The amount of thickener and/or stabiliser in the composition or in the outer phase may be from 0.00001 wt % to 30 wt %, or 0.001 wt % to 30 wt %, such as from 0.01 wt % to 10 wt %, preferably from 0.1 wt % to 2 wt %, such as from 0.1 wt % to 1 wt %.

Compounds of Formula (I) (e.g. TPGS)

The compound of Formula (I) has the structure:

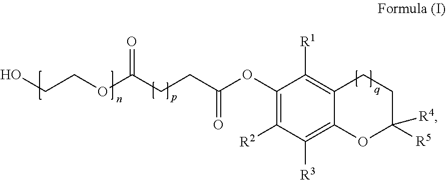

wherein R¹, R², R³ and R⁴ are each independently H or a C1-6 hydrocarbon group that is optionally substituted by one or more Y group; R⁵ is a C6-30 hydrocarbon group; n is from 3 to 100; p is from 0 to 6; q is from 0 to 3; and Y is selected from the list consisting of ether, ester, sulfone, sulfoxide, amide, amine e.g. secondary amine and tertiary amine), boronate ester, ketone and aldehyde.

Hydrocarbon groups may include alkyl, alkenyl, alkynyl and/or aryl groups. Preferably each hydrocarbon group is an alkyl group.

Preferably R¹, R², R³ and R⁴ are each independently H or a C1-3 hydrocarbon group that is optionally substituted by one or more Y group. For example, R¹, R², R³ and R⁴ may each independently be H or a C1-3 hydrocarbon group that is optionally substituted by one Y group; more preferably R¹, R², R³ and R⁴ are each independently be H or a C1-3 hydrocarbon group that is not substituted, for example H or a C1-3 alkyl group. More preferably R¹, R², R³ and R⁴ are each independently a C1, C2 or C3 alkyl group. Most preferably R¹, R², R³ and R⁴ are each C1 (i.e. methyl) groups.

n is from 3 to 100. For example, n may be from 5 to 100, preferably from 10 to 100, or from 15 to 100, such as from 20 to 100. n may be from 5 to 80, such as from 5 to 60, preferably from 5 to 40, such as from 5 to 35, or from 5 to 30, for instance from 5 to 25. n may be from 5 to 60, such as from 10 to 40, or from 15 to 30.

p is from 0 to 6. For example, p may be from 0 to 4, such as from 0 to 3, or from 0 to 2. Preferably p is 0, 1 or 2, most preferably 1.

q is from 0 to 3, such as from 0 to 2. Preferably q is 0 or 1; most preferably q is 1.

Preferably Y is selected from the list consisting of ether, ester, sulfone, sulfoxide, amide, and ketone.

Preferably R⁵ is a C6-26 hydrocarbon group, such as a C6-22 hydrocarbon group, or a C6-18 hydrocarbon group, for example a C6-16 hydrocarbon group. R⁵ may be a C8-hydrocarbon group, such as a C10-30 hydrocarbon group, or a C12-30 hydrocarbon group, such as a C14-30 hydrocarbon group or a C16-30 hydrocarbon group. R⁵ may be a C8-26 hydrocarbon group or a C12-20 hydrocarbon group. Preferably the hydrocarbon group of R⁵ is an alkyl group, for example a C8-26 alkyl group or a C12-alkyl group.

A compound of Formula (I) may be represented by Formula (II):

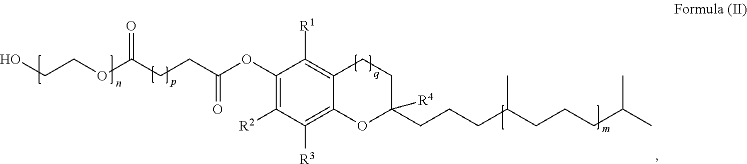

wherein m of Formula (II) is an integer from 0 to 4, preferably from 1 to 3, and most preferably 2.

Options described herein in relation to Formula (I) apply equally to Formula (II) unless inconsistent.

In one embodiment:
the compound is of Formula (I);
R¹, R², R³ and R⁴ are each independently H or a C1-3 alkyl group that is optionally substituted by one Y group;
R⁵ is a C6-30 hydrocarbon group;
n is from 3 to 100;
p is from 0 to 2;
q is 0 or 1; and
Y is selected from the list consisting of ether, ester, sulfone, sulfoxide, amide, amine e.g. secondary amine and tertiary amine), boronate ester, ketone and aldehyde.

In one embodiment:
the compound is of Formula (I);
R¹, R², R³ and R⁴ are each independently H or a C1-3 alkyl group;
R⁵ is a C6-30 hydrocarbon group;
n is from 10 to 40;
p is from 0 to 2; and
q is 0 or 1.

In one embodiment:
the compound is of Formula (II);
R¹, R², R³ and R⁴ are each independently H or a C1-3 alkyl group;
m is from 1 to 3;
n is from 10 to 40;
p is from 0 to 2; and
q is 0 or 1.

In one embodiment:
the compound is of Formula (II);
R¹, R², R³ and R⁴ are each independently C1 or a C2 alkyl group;
m is from 1 to 3;
n is from 15 to 30;
p is 1; and
q is 1.

Most preferably the compound of Formula (I) is tocopheryl polyethylene glycol succinate (TPGS also known as tocofersolan or tocophersolan).

TPGS is a water-soluble vitamin E conjugate. In vivo, enzymatic cleavage of TPGS can provide tocopherol (vitamin E).

Preferably the polyethylene glycol (PEG) unit, as shown in the brackets of the structures of TPGS, has a number average molecular weight of from 100 to 5000, such as from 500 to 3000, preferably from 800 to 2500, such as 800 to 1500, most preferably 1000. TPGS with a PEG group average (e.g. number average) molecular weight of 1000 g mol⁻¹ is known as “TPGS 1000”, e.g. “d-α-tocopheryl polyethylene glycol 1000 succinate”, and is commercially available (CAS 9002-96-4). TPGS 2000, having a PEG group average molecular weight of 2000 g mol⁻¹ is also commercially available.

The compound of Formula (I), e.g. TPGS, may be present in any stereoisomeric or tautomeric form. Preferably the compound is in the D-α-stereoisomer form:

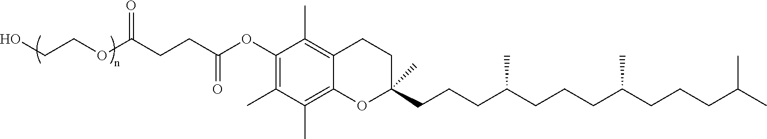

D-α-form of TPGS

The TPGS (α-tocopherol polyethylene glycol succinate) may contain any polyethylene glycol chain length. For example the TPGS may be TPGS 200, TPGS 300, TPGS 400, TPGS 750, TPGS 1000, TPGS 1500, TPGS 2000, TPGS 3000 and/or TPGS 4000. Preferably the TPGS is TPGS 1000.

Preferably, the aqueous (outer) phase further comprises a water-soluble agent. The water-soluble agent may be PHMB (e.g. buffered PHMB). Optionally, the water-soluble agent may comprise hyaluronic acid, insulin, ascorbic acid and/or L-ascorbic acid, for example insulin, ascorbic acid and/or L-ascorbic acid. The aqueous TPGS outer phase may additionally comprise polysorbate.

The amount of the compound of Formula (I), such as TPGS (e.g. TPGS 1000), in the composition or in the outer phase may be 0.001 wt % or more, such as 0.01 wt % or more, or 0.1 wt % or more, such as 0.5 wt % or more, or 1 wt % or more, preferably 2 wt % or more, such as 4 wt % or more, or 5 wt % or more, for example 8 wt % or more. The amount of the compound of Formula (I) in the composition or in the outer phase may be 40 wt % or less, such as 30 wt % or less, or 20 wt % or less, such as 15 wt % or less, or 12 wt % or less, such as 11 wt % or less. The amount may be from 0.001 wt % to 40 wt %, such as from 0.1 wt % to 40 wt %, or 0.5 wt % to 30 wt %, such as from 1 wt % to 20 wt %, or from 5 wt % to 15 wt %.

### Combinations

The composition may contain a retinoid, such as retinol or retinaldehyde, curcumin and/or cannabidiol (preferably retinol, e.g. in an amount of 0.001 wt % or more, such as 0.1 wt % or more). The composition may contain, as a solvent, water, ethanol and/or polysorbate. The (average) micelle size may be from 5 to 50 nm (e.g. from 5 to 20 nm, or from 5 to 15 nm. In such a composition, the amount of the compound of Formula (I), e.g. TPGS, may be from 1 wt % to 30 wt %.

The composition may contain a retinoid, such as retinaldehyde or retinol, curcumin and/or cannabidiol in an amount of 5 wt % or less. The composition may contain, as a solvent, water, ethanol and/or polysorbate. The (average) micelle size may be 80 nm or less (such as 50 nm or less, or from 5 to 80 nm). In such a composition, the amount of the compound of Formula (I), e.g. TPGS, may be from 1 wt % to 30 wt %.

The composition may contain a retinoid, such as retinaldehyde or retinol, curcumin and/or cannabidiol in an amount of 2 wt % or less. The composition may contain, as a solvent, water, ethanol and/or polysorbate. The (average) micelle size may be 20 nm or less (such as 15 nm or less, or from 5 to 15 nm). In such a composition, the amount of the compound of Formula (I), e.g. TPGS, may be from 1 wt % to 30 wt %.

The composition may contain a retinoid, such as retinaldehyde or retinol, curcumin and/or cannabidiol in an amount of 1 wt % or less, or 0.5 wt % or less. The composition may contain, as a solvent, water, ethanol and/or polysorbate. The (average) micelle size may be 10 nm or less (such as from 5 to 10 nm). In such a composition, the amount of the compound of Formula (I), e.g. TPGS, may be from 1 wt % to 30 wt %.

The composition may contain a retinoid, such as retinaldehyde or retinol, curcumin and/or cannabidiol (preferably retinol, e.g. in an amount of 0.001 wt % or more, such as 0.1 wt % or more). The composition may contain PHMB and/or xanthan gum (e.g. in an amount of 0.00001 wt % to 10 wt %, such as 0.01 to 20 wt %, of the composition and/or the outer phase). The composition may contain, as a solvent, water, ethanol and/or polysorbate. The (average) micelle size may be from 5 to 50 nm (e.g. from 5 to nm, or from 5 to 15 nm. In such a composition, the amount of the compound of Formula (I), e.g. TPGS, may be from 1 wt % to 30 wt %.

### Formulation

The micellar composition may constitute a cosmetic formulation. Preferably, the micellar composition is in the form of a cream, lotion, gel, semi-solid, dispersion, suspension, foam, mousse or spray.

The micellar composition may be a food or beverage composition.

The diameter of the micelles (e.g. mean diameter) may be measured by dynamic light scattering (DLS) and/or transmission electron microscopy (TEM).

The micelles may have a (mean) diameter of 45 nm, or less, 40 nm or less, 35 nm or less, 30 nm or less, 25 nm or less, 24 nm or less, 23 nm or less, 22 nm or less, 21 nm or less or 20 nm or less, or 15 nm or less, or 10 nm or less; and/or the micelles may have mean diameter of 1 nm or more, 2 nm or more, 5 nm or more, 8 nm or more, 10 nm or more, 12 nm or more, 14 nm or more, 15 nm or more, 17 nm or more, 18 nm or more, nm or more.

The composition may comprise micelles having a mean diameter of 5 to 45 nm, 8 to nm, 10 to 30 nm, 15 to 25 nm, 21 to 25 nm, or 22 to 24 nm. The composition may comprise micelles having a mean diameter of 14 to 16 nm, such as 15 nm. The composition may comprise micelles having a mean diameter of 18 to 20 nm.

The composition may comprise micelles with a size (e.g. diameter) distribution such that the proportion of micelles having a size of 100 nm or less is 70% or more, such as 80% or more, or 90% or more of the micelles (by number). For example, the proportion may be 99.99% or less, such as 99.9% or less, or 99% or less, for example 98% or less. The proportion of micelles having a size of 100 nm or less may be from 70% to 99.99%, such as from 80% to 99.9%, or from 90% to 99%.

The composition may comprise micelles with a size (e.g. diameter) distribution such that the proportion of micelles having a size of 50 nm or less is 70% or more, such as 80% or more, or 90% or more of the micelles (by number). For example, the proportion may be 99.99% or less, such as 99.9% or less, or 99% or less, for example 98% or less. The proportion of micelles having a size of 50 nm or less may be from 70% to 99.99%, such as from 80% to 99.9%, or from 90% to 99%.

The composition may comprise micelles with a size (e.g. diameter) distribution such that the proportion of micelles having a size of 20 nm or less is 70% or more, such as 80% or more, or 90% or more of the micelles (by number). For example, the proportion may be 99.99% or less, such as 99.9% or less, or 99% or less, for example 98% or less. The proportion of micelles having a size of 20 nm or less may be from 70% to 99.99%, such as from 80% to 99.9%, or from 90% to 99%.

The composition may comprise micelles with a size (e.g. diameter) distribution such that the proportion of micelles having a size of 10 nm or less is 70% or more, such as 80% or more, or 90% or more of the micelles (by number). For example, the proportion may be 99.99% or less, such as 99.9% or less, or 99% or less, for example 98% or less. The proportion of micelles having a size of 10 nm or less may be from 70% to 99.99%, such as from 80% to 99.9%, or from 90% to 99%.

Size distribution may be described with reference to volume, intensity or number. In the present specification “mean diameter” refers to a volume mean particle diameter, unless otherwise stated.

The (mean) diameter may refer to the empty or unloaded micelles, i.e. without a cargo.

The (mean) diameter may refer to loaded micelles, i.e. with a cargo.

### Method of Production

The method of producing the micellar composition comprises: providing hydrophobic phase comprising an organic solvent (e.g. ethanol and/or polysorbate); adding the hydrophobic phase into aqueous dispersion of the compound of Formula (I), e.g. TPGS; and optionally removing at least a portion of the organic solvent.

The method may be performed at a temperature of around room temperature (e.g. from 5° C. to 60° C., such as from 5° C. to 40° C., or from 10° C. to 30° C., such as from 15° C. to 25° C.

Ethanol may be removed to yield the micellar composition. However, it is noted that micelles are typically formed before the ethanol is removed, so removal of ethanol is not required for micelles to be formed.

Polysorbate may not require removal to yield the micellar composition. However, some or all can be removed with a reverse filtration process.

Preferably, the hydrophobic phase is added into the aqueous dispersion of the compound of Formula (I), e.g. TPGS, at a concentration in the range 1% to 20%. Alternatively, the hydrophobic phase may be in the range 2% to 19%, 3% to 18%, 4% to 17%, 5% to 16%, 6% to 15%, 7% to 14%, 8% to 13%, 9% to 12%, 2% to 20%, 3% to 20%, 4% to 20%, 5% to 20%, 6% to 20%, 7% to 20%, 8% to 20%, 9% to 20%, 10% to 20%, 11% to 20%, 12% to 20%, 13% to 20%, 14% to 20%, 15% to 20%, 16% to 20%, 17% to 20%, 18% to 20%, 19%, 19.5% or 20%. More preferably, the hydrophobic phase is added into the aqueous dispersion of the compound of Formula (I), e.g. TPGS, at a concentration of 10%. Percentages given here may be by volume.

The method may additionally comprise separating the micelles of a desired particle size.

A range of permissible solvents may be used in conjunction with the invention (e.g. the methods thereof), for example to solubilise bio-active agents for encapsulation in micelles depending on the chosen application. Suitable solvents will depend on the bio-active being incorporated into the micellar composition and may be selected accordingly. If it is intended to incorporate one bio-active in the micellar composition, for example retinol then an appropriate solvent will be one in which the chosen bio-active is soluble. Depending on the desired application, a mixture of miscible solvents may be used. Similarly, if it is intended to incorporate more than one bio-active in the micellar composition, for example retinol and ascorbyl palmitate then an appropriate solvent will be one in which both the chosen bio-actives are soluble.

The organic solvent may be a volatile organic solvent and may include, but is not limited to one selected from; GRAS solvents (e.g. acetic acid, anisole, butyl butyrate, 1,3-butylene glycol, ethanol, ethyl acetate, ethyl benzoate, ethyl butyrate, ethyl decanoate, ethyl formate, ethyl hexanoate, ethyl lactate, ethylene dichloride, glycerin, glycerol, glyceryl monooleate, glyceryl palmitostearate, isoamyl acetate, isobutyl acetate, isopropyl acetate, isopropyl alcohol, isopropyl citrate, lactic acid, linoleic acid, methyl acetate, octanoic acid, propionic acid, propyl acetate, stearic acid, water, ethyl vanillin, limonene) or ethanol. The organic solvent is preferably an alcohol. More preferably the organic solvent is a C2-C6 carbon chain length alcohol, such as ethanol, iso-propanol, n-propanol, iso-butanol, s-butanol, t-butanol and/or n-butanol. The organic solvent is preferably ethanol. Preferably the solvent is used in combination with a thickener/stabiliser, such as a thickener/stabiliser selected from the lists above. More preferably, ethanol is used in combination with polysorbate and/or polyvinyl caprolactam-polyvinyl acetate-polyethylene glycol graft copolymer.

Preferably, at least a portion of the volatile organic solvent is removed by subjecting the micellar composition to a vacuum or heating or sonification or a combination thereof.

Preferably, the volatile organic solvent is removed by subjecting the micellar composition to heating and a vacuum. In methods where heating is applied, either alone or in combination with other methods such as application of a vacuum, preferably, the temperature used to remove the volatile organic solvent will be between 50° C. and 60° C.

The concentration of organic solvent in the composition may additionally or alternatively be reduced by dilution and/or (tangential) trans flow filtration, which avoid the use of heat to remove solvents.

The combined steps of dilution and trans flow filtration may allow the removal of a higher proportion of the organic solvent (e.g. ethanol) from the micelles, for example to reach a level that exhibits lower toxicity and/or sensitivity for the subject (e.g. their skin). For example, the level of organic solvent (e.g. ethanol) may be reduced to 4 vol % or less, such as 2 vol % or less, or 1 vol % or less, such as 0.5 vol % or less, such as 0.1 vol % or less, or 0.001 vol % or less of the composition. Low levels of alcohols such as ethanol (e.g. 1 vol % or less) may assist with cell replication, providing the compositions with further benefits.

Owing to the possibility to reduce the organic solvent (e.g. ethanol) content to a very low level, the composition may be, or be incorporated into, a beverage.

An aqueous liquid (e.g. water, phosphate-buffered saline) may be added to the composition following the removal of the organic solvent (e.g. ethanol). The amount of the aqueous liquid added may be 5 vol % or more, such as 10 vol % or more, or 20 vol % or more, such as 40 vol % or more of the resulting composition (following the addition), such as from 10 vol % to 99 vol %, or from 20 vol % to 95 vol %, or from 20 vol % to 80 vol %.

The removal of organic solvent and/or addition of aqueous liquid may be used to control the concentration of the compound of Formula (I), e.g. TPGS, and/or any biologically active agents (e.g. retinol), and may be varied according to the particular application.

In accordance with the invention, the loading capacity of micelles is measured as w/w of the compound of Formula (I), e.g. TPGS, and the cargo (e.g. bio-active (therapeutic) agent). In the case of micelles “high loading capacity” is in the range 0.1-20% inner oil phase. Accordingly, loading capacity may be in the range 0.1-20% (w/w), 1-20% (w/w), 2-20% (w/w), 3-20% (w/w), 4-20% (w/w), 5-20% (w/w), 6-20% (w/w), 7-20% (w/w), 8-20% (w/w), 9-20% (w/w), 10-20% (w/w), 11-20% (w/w), 12-20% (w/w), 13-20% (w/w), 14-20% (w/w), 15-20% (w/w), 16-20% (w/w), 17-20% (w/w), 18-20% (w/w) or 19-20% (w/w). Alternatively, loading capacity of the micelles may be in the range 0.1-20% (w/w), 1-19% (w/w), 1-18% (w/w), 1-17% (w/w), 1-16% (w/w), 1-15% (w/w), 1-14% (w/w), 1-13% (w/w), 1-12% (w/w), 1-11% (w/w), 1-12% (w/w), 1-11% (w/w), 1-10% (w/w), 1-9% (w/w), 1-8% (w/w), 1-7% (w/w), 1-6% (w/w), 1-5% (w/w), 1-4% (w/w), 1-3% (w/w) or 1-2% (w/w). Preferably, loading capacity of the micelles may be in the range 1-20% (w/w), 2-19% (w/w), 3-18% (w/w), 4-17% (w/w), 5-16% (w/w), 6-15% (w/w), 7-14% (w/w), 8-13% (w/w), 9-12% (w/w), 10-11% (w/w).

The composition may include a biologically active agent (e.g. a retinoid, such as retinol or retinal, curcumin and/or cannabidiol) and be applied to the skin of a subject (in an amount sufficient) to provide a dose of the biologically active agent, per cm² of skin, of Ing or more, such as 10 ng or more, or 100 ng or more, for example 1 μg or more, such as 10 μg or more, or 20 μg or more. The dose per cm² of skin may be 2000 μg or less, or 1500 μg or less, preferably 1000 μg or less, such as 800 μg or less, or 700 μg or less, or 600 μg or less. More preferably the dose of biologically active agent per cm² of skin is 400 μg or less, such as 200 μg or less, or 100 μg or less, yet more preferably 75 μg or less, such as 50 μg or less, more preferably 30 μg or less, or 20 μg or less, especially 15 μg or less, or 10 μg or less, such as 8 μg or less, or 5 μg or less. The dose per cm² of skin may be from 1 ng to 2000 μg, such as from 10 ng to 1000 μg, or from 10 ng to 800 μg. Preferably the dose per cm² of skin is from 1 ng to 100 μg, such as from Ing to 50 μg, or from Ing to 15 μg, such as from 1 ng to 10 μg.

### BRIEF DESCRIPTION OF THE FIGURES

The invention will now be described in detail with reference to examples and with reference to the accompanying drawings, in which:

FIG. 1 is a schematic diagram to illustrate transdermal penetration;

FIG. 2 shows an experimental set-up for the dermal absorption studies of micellar retinol;

FIG. 3 shows the results of a cytotoxicity study;

FIG. 4 shows a DLS result and a TEM micrograph of a micellar retinol formulation on (a) day 1 of production and (b) after 150 days of storage;

FIG. 5 (a) DLS result of the micellar retinol formulation on day 0 of production (b) an SEM image of the micellar retinol formulation on day 0 of production, and (c) DLS result of the micellar retinol formulation after 1 year of storage;

FIG. 6 shows the cumulative amount of retinol detected in the receptor fluid of the diffusion cell system over 24 hours, where data are presented as mean±s.d (N=3,n=2);

FIG. 7 shows cumulative amount of retinol detected in the receptor fluid of the diffusion cell system over 80 hours, where data are presented as mean±s.d (N=3,n=2);

FIG. 8 shows (a) the cross-section of the Strat-M membrane after the retinol micelle penetration experiment and (b) an enlarged view of the bottom side of the Strat-M membrane after the experiment;

FIG. 9 shows (a) the confocal image of the retinol micelles in the membrane, and (b) an enlarged view of retinol gradients within the skin model after the diffusion study for the retinol formulation;

FIG. 10 is a comparison of skin penetration of micellar retinol and free retinol;

FIG. 11 shows human skin results for retinol penetration using Raman spectroscopy;

FIGS. 12 to 21 show DLS data for compositions of the invention; and

FIGS. 22 to 24 show additional data for compositions of the invention.

### EXAMPLES

### Skin Penetration of a Micellar Antiaging Retinol Formulation

Retinol (Vitamin A) is widely used as an antioxidant and antiaging compound in various cosmeceuticals, having an increasing trend in the cosmetic and personal care market. Retinol exhibits low stability and is prone to degradation, therefore encapsulation of retinol within a vehicle would enhance its efficacy. It has been found that microemulsions, known also as micellar emulsions, are able to improve the physicochemical stability of sensitive compounds, offering at the same time slow/controlled release of the active ingredient. Slower/controlled release of the active ingredient can assist with lower toxicity/irritation implications. Micellar emulsions have small particle size (<100 nm) and high surface area. It has been found that this makes them promising candidates for transdermal delivery of actives.

The micellar retinol formulation described herein benefits from small particle size, long-term stability, controlled release and lower irritation implications.

Particle size can be determined using a Malvern Instruments Zetasizer (Zetasier 3000; Malvern Instruments Co. Ltd.). 1 mL sample is placed in a polystyrene disposable cuvette and exposed to laser diffraction at an angle of 90° to determine the size of the micelles.

Aim: in this work, the transdermal penetration of the micellar retinol formulation has been evaluated. Referring to FIG. 1 there is shown a droplet of micellar composition for application to the skin 12.

### Materials

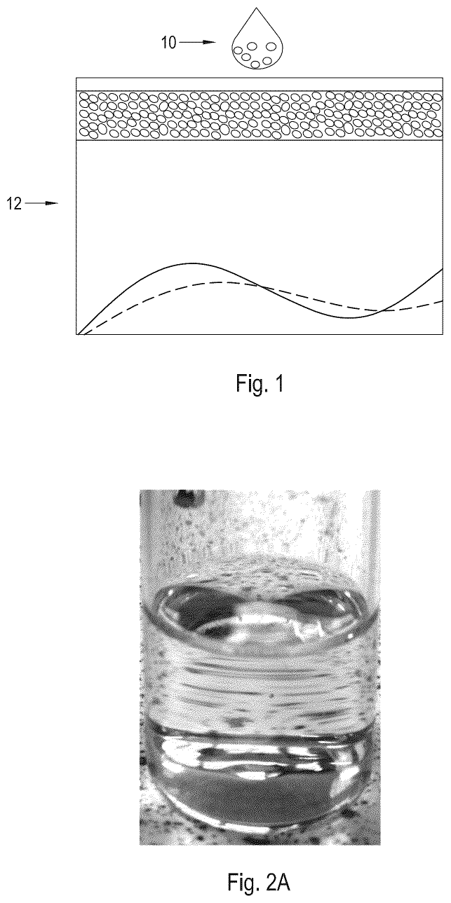

A 5% micellar retinol composition (FIG. 2A) was prepared following the method of GB2550346B i.e. by dissolving Retinol in an organic solvent (ethanol) to provide a hydrophobic phase; b. adding the hydrophobic phase into aqueous TPGS; and c. removing ethanol to yield the micelles.

### Cytotoxicity of Retinoids

The cytotoxicity of retinol was determined.

Experiment 1: A solution of retinol (20 mg/mL) in ethanol (absolute) was prepared. The solution was diluted to 2 mg/mL, forming a light yellow emulsion. The emulsion was diluted in optiMem to provide concentrations of 5, 2.5, 1.25, 0.625 and 0.31 μg/mL retinol. Untreated and detergent controls were included for comparison. Using a Presto Blue assay, adult human dermal fibroblast (HDFa) cells (5 k per well) were exposed to the solutions for 24 h.

Experiment 2: A solution of retinol (20 mg/mL) in 1,2-propanediol was prepared. The solution was diluted to 2 mg/mL, forming an off-white emulsion. The emulsion was diluted in optiMem to provide concentrations of 10, 5, 2.5, 1.25, 0.625 and 0.31 μg/mL retinol. Untreated and detergent controls were included for comparison. Using a WST-1 probe, HDFa cells (5 k per well) were exposed to the solutions for 24 h.

Experiment 3: A solution of retinol (20 mg/mL) in ethanol (absolute) was prepared. The solution was diluted to 2 mg/mL, forming an light yellow emulsion. The emulsion was diluted in optiMem to provide concentrations of 10, 5, 2.5, 1.25, 0.625 and 0.31 μg/mL retinol. Untreated and detergent controls were included for comparison. Using a WST-1 probe, HDFa cells (5 k per well) were exposed to the solutions for 24 h.

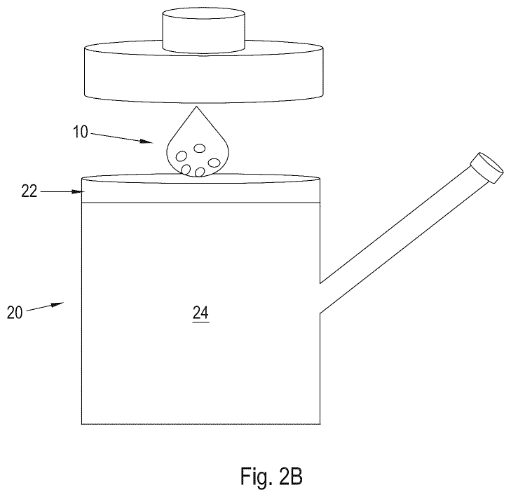

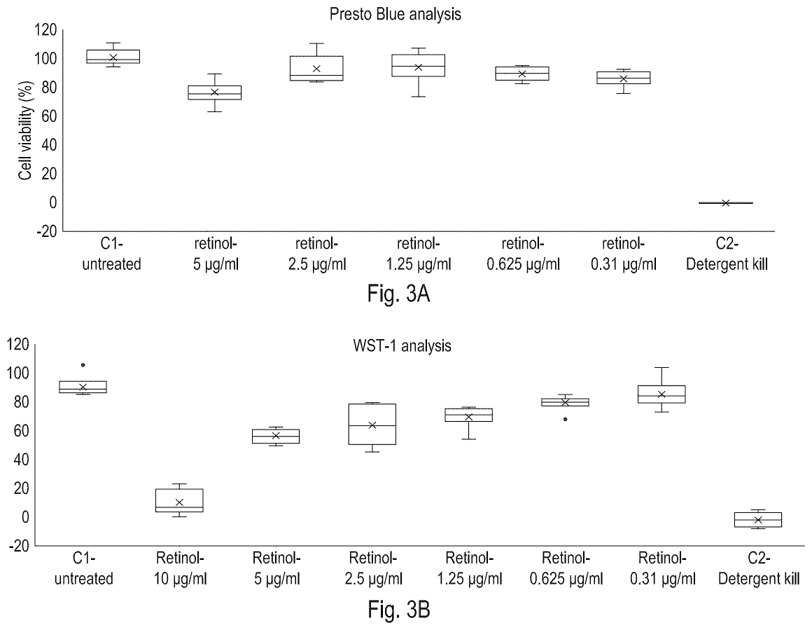

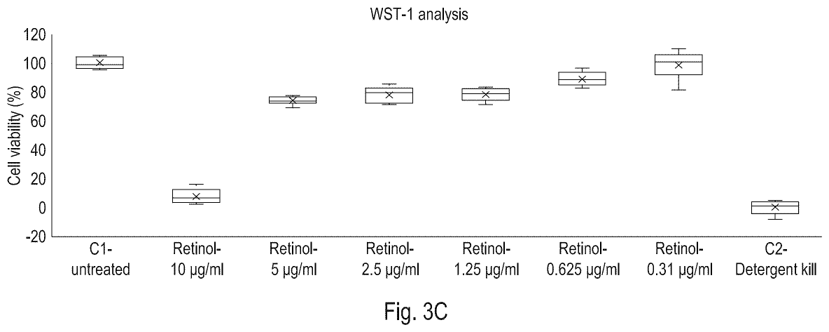

FIG. 3 shows the results of this cytotoxicity study. FIG. 3A, FIG. 3B and FIG. 3C show the results for Experiments 1, 2 and 3 respectively.

The results show that retinol is cytotoxic to the HDFa cell line at concentrations of 10 μg/mL and above. However, where the concentration is controlled to be below 10 μg/mL, the retinol showed only little, if any, cytotoxicity.

### Long-Term Stability

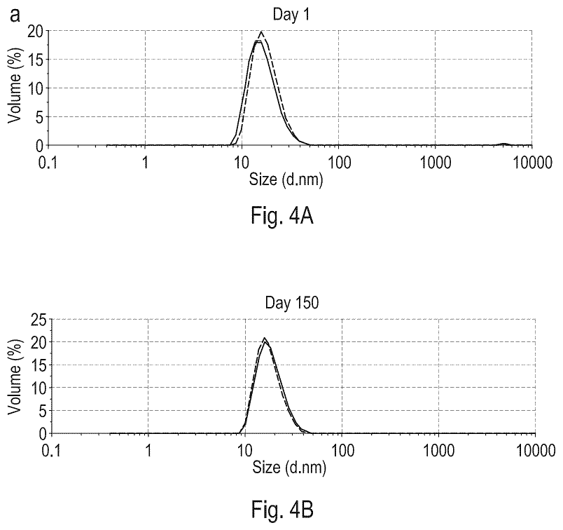

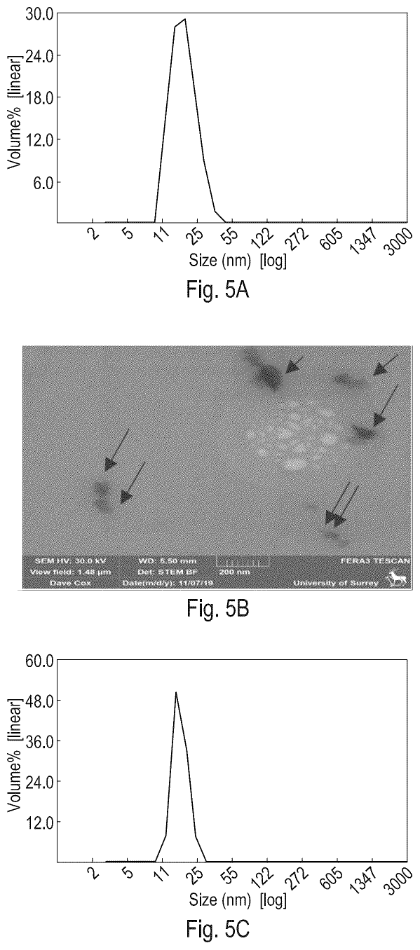

Long-term stability of the micellar retinol is demonstrated by FIGS. 4 and 5, and tabulated below. FIG. 5B shows arrows pointing to the micelles.

An aliquot of the micellar solution was diluted 5000 times in ultrapure H₂O (18.5 MΩ·cm) and the free retinol content was measured with UV spectroscopy at 325 nm wavelength. A calibration curve for a series concentrations of retinol solutions has been previously generated to enable the accurate quantification of the free retinol in solution. The encapsulation efficiency (EE) was calculated using the following equation:

$$EE \% = \frac{Total amount of retinol in the micelles - Amount of free retinol}{Total amount of retinol in the micelles} \times 10$$

| Timepoint | Particle | Encapsulation |
|---|---|---|
| (days) | size (nm) | efficiency ( %) |
| 1 | 22.9 ± 0.8 | 84.5 ± 0.8 |
| 150 | 23.9 ± 0.8 | 82.8 ± 0.2 |

The particle size of the micellar retinol formulation is uniform and also has long-term stability with a shelf life more than 18 months.

### Retinoid Distribution

As illustrated in FIG. 2B modified Franz diffusion cells 20 incorporating a Strat-M membrane 22 was used as skin model to evaluate the micellar formulation penetration. The Strat-M membranes were mounted on the diffusion cells (Phoenix™ DB-6, Teledyne Hanson Research), which had 1 cm² effective diffusion area. The receptor chamber was filled with 20% v/v ethanol in Phosphate Buffered Saline (PBS; Merk, Germany)+0.01% sodium azide (NaN₃; Merk, Germany) to enable the solubilization of retinol. The receptor fluid was continuously stirred at 500 rpm, heated at 37.5±1° C. to maintain a constant temperature on the membranes' surface of 32.5±1° C. Then 160 mg/cm² of the 5% micellar retinol formulation (total dosage of 8 mg/cm² of retinol) were applied to the skin surface to allow a total penetration duration of 24 hr. Receptor fluid samples were collected at frequent timepoints and analysed by HPLC (Agilent 1100/1200 Chemstation, USA, and Eclipse XDB-C18 column, Agilent, UK, mobile phase:methanol:water (90:10) with a flow rate of 1.5 mL/min. UV absorption was read at 325 nm. Retention time for retinol was 2.6 min. All procedures were conducted at 25° C.). At the end of the experiment, the Strat-M® membrane's surface was cleaned with a cotton swab twice. Afterwards, the Strat-M® membrane was cut in small pieces and immersed in pure ethanol solution. The solution was homogenized (Fisher brand 850, Fisher Scientific) at 5000 rpm for 2 mins. The experiments were performed in triplicate (n=3). The distance from the top side of the Strat-M membrane to the bottom side is 300 μm.

Spatial distribution of retinol within the skin model has been assessed with SEM and Confocal Scanning Laser Microscopy (CSLM).

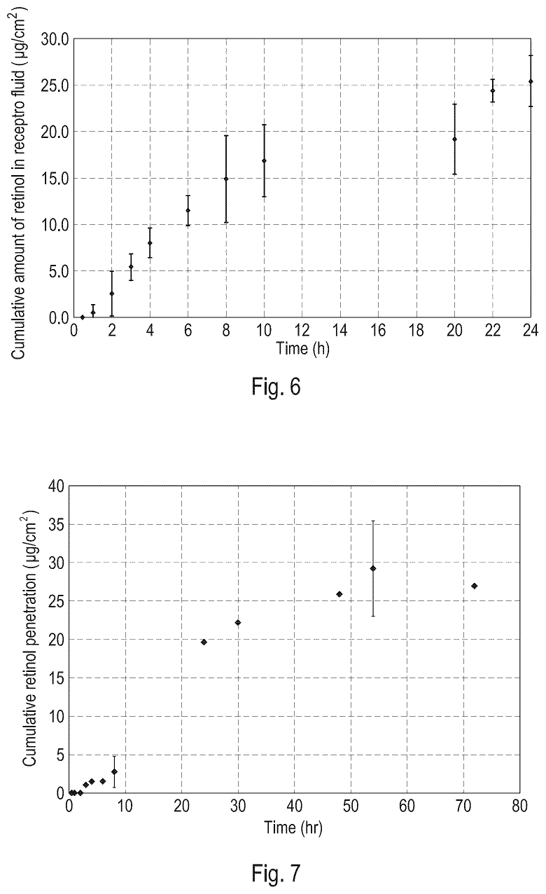

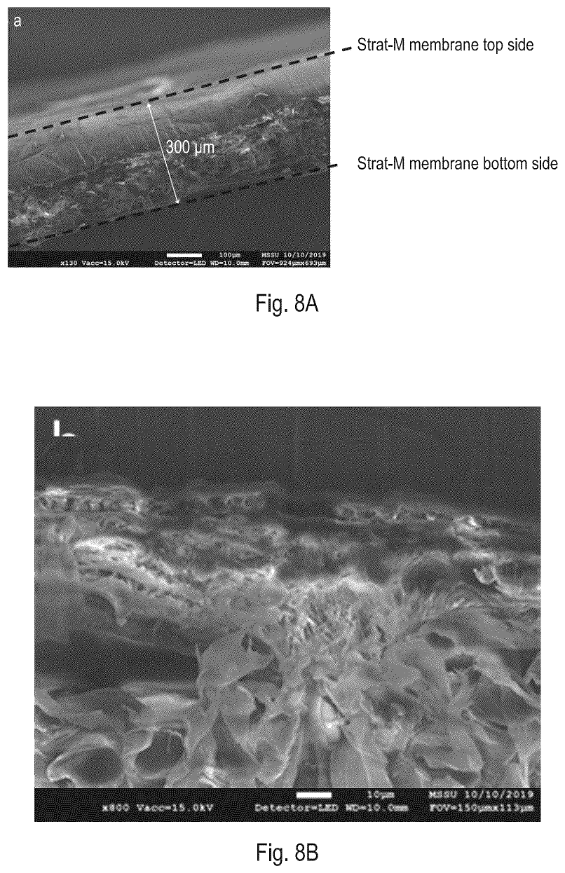

Retinol accumulation in the receptor fluid is shown in FIGS. 6 and 7. Low percentage (0.32%) of the total retinol was detected in the receptor fluid after 24 hours.

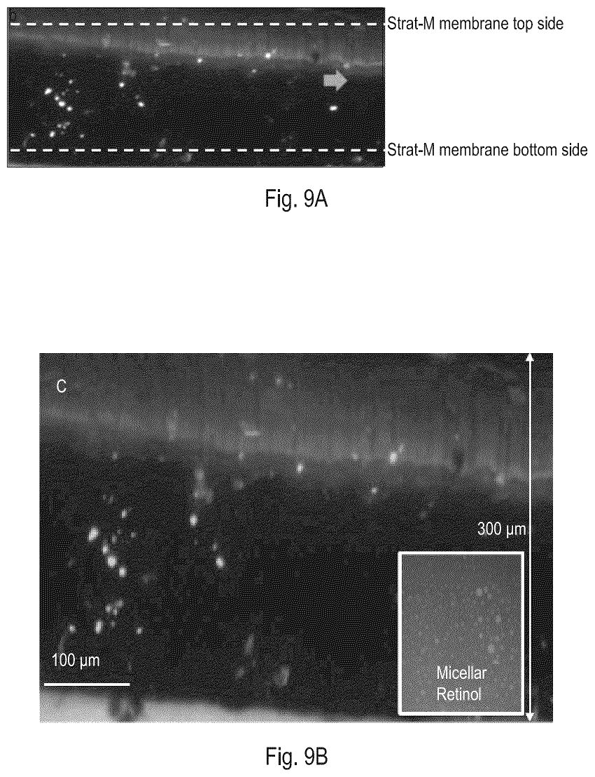

Spatial distribution of retinol penetration is the skin model is shown in FIGS. 8 and 9. Retinol gradients were detected within the skin model. The retinol micelles were able to penetrate the membrane's surface, which is equivalent to the stratum corneum in human skin. The retinol was mainly retained and distributed in the upper layers of the membrane, equivalent to the epidermis of skin.

### Comparison to Free Retinol

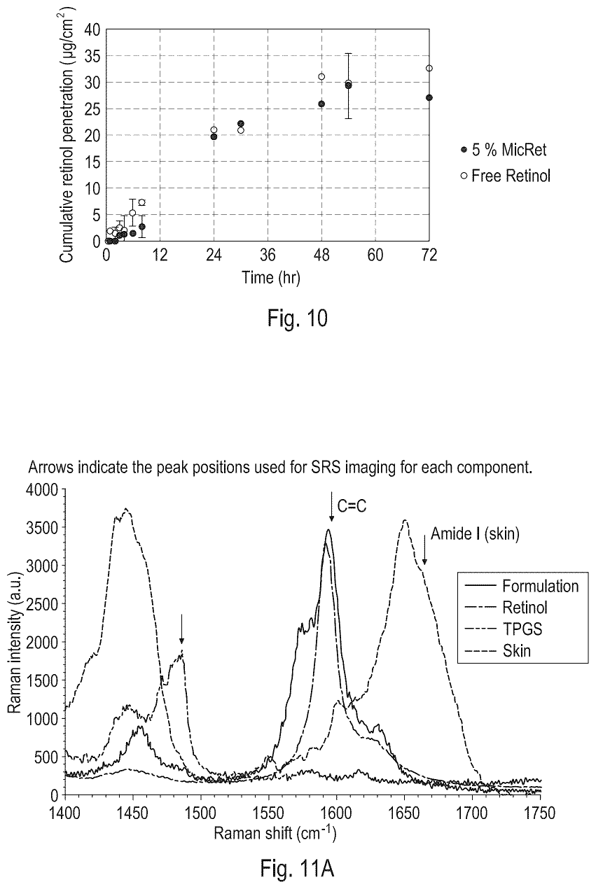

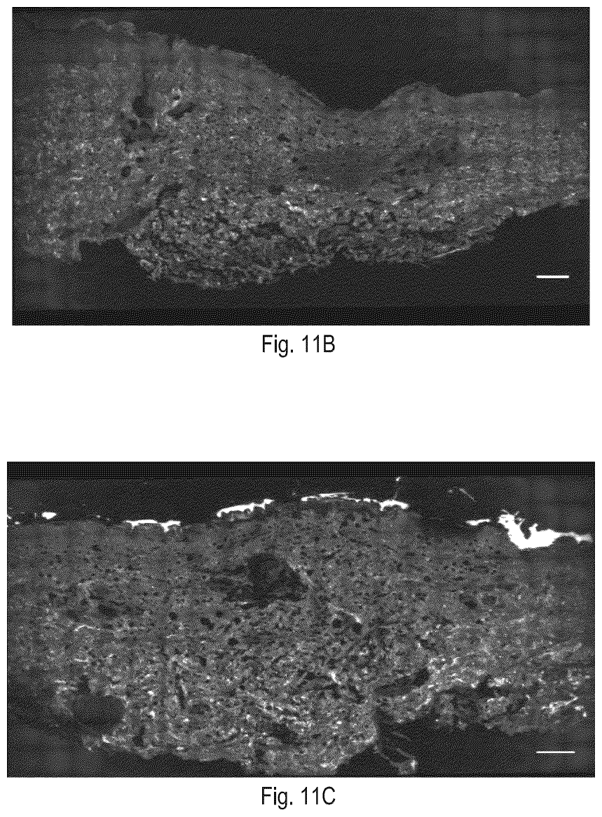

The penetration of micellar 5% retinol was compared that of a 5% aqueous retinol in ethanol solution (i.e. free retinol). FIG. 10 shows the results of this study.

The micellar retinol was found to perform as well as free retinol in ethanol over time. This is particularly beneficial considering that the micellar retinol simultaneously provides significant stability benefits described above.

### SUMMARY

In this work, the topical penetration efficacy of the micellar retinoid (e.g. retinol) formulation has been evaluated using a Strat-M membrane as a skin model, assessing its potential use for transdermal delivery of retinoids such as retinol.

The penetration studies using Franz-diffusion cell revealed that, after 24 hours, 3.6% of the applied retinol penetrated into the skin model and only 0.3% was detected in the receptor fluid.

Due to the similarities of the Strat-M membranes with human skin, this data illustrates that the micellar retinoid (e.g. retinol) formulation will provide the combined benefits of readily being able to penetrate into human skin, and only a minimal amount entering the blood stream.

In addition, the micellar retinoid (e.g. retinol) formulation was found to have excellent long-term stability.

These findings showed the great potential of the micellar retinoid (e.g. retinol) as an antiaging formulation for transdermal skin delivery.

### Additional Data

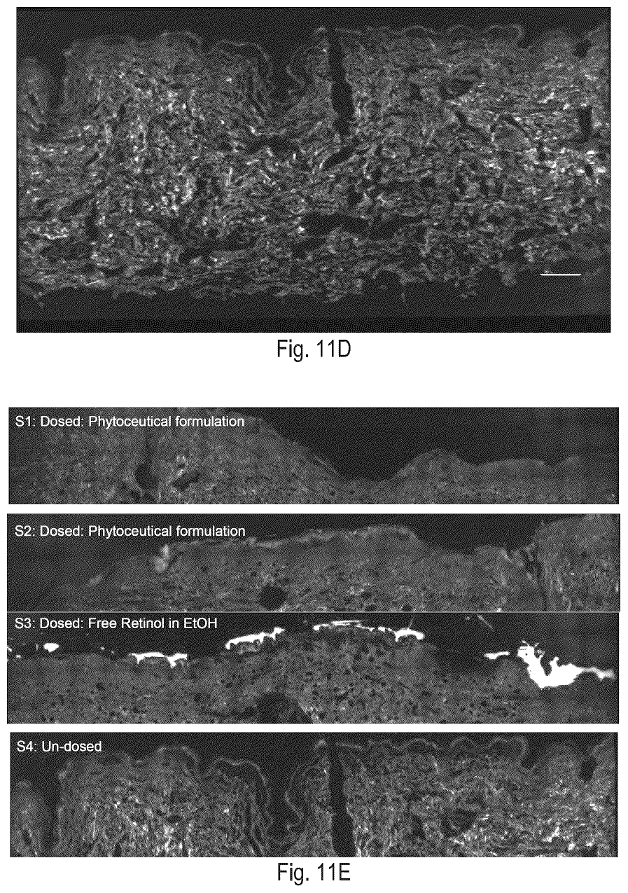

FIG. 11 shows human skin results for retinol penetration using Raman spectroscopy. SRS microscopy was successful in visualising retinol distribution in skin samples further supporting the delivery of retinol into skin, from the C═C vibrational band, with micellar retinol of the invention and comparable to free retinol in ethanol, although images at this wavenumber also contain some contributions from endogenous species in the skin. These endogenous signals were particularly associated with the basal layer of the dermis, consistent with the presence of melanin/melanocytes.

FIG. 11A shows wavenumbers used for SRS imaging in FIGS. 11B-11E. Red: SRS for C═C (retinol); Blue: SRS for Amide I (skin); Green: SHG for Collagen.

FIG. 11B shows skin dosed with the micellar composition of the invention. FIG. 11C shows skin dosed with free retinol in EtOH. FIG. 11D shows un-dosed skin. FIG. 11E directly compares the images in FIGS. 11B-11D.
10% aqueous TPGS micelles were prepared with and without PHMB (0.1 wt %) and DLS was determined. The results are summarised below:

| | DLS Peak (nm) | | |
|---|---|---|---|
| Micelle formation | Intensity | Number | Volume |
| Empty micelles | 9.577 | 6.103 | 7.364 |
| PHMB added before homogenisation | 9.686 | 6.510 | 7.730 |
| PHMB added after homogenisation | 9.909 | 6.421 | 7.716 |

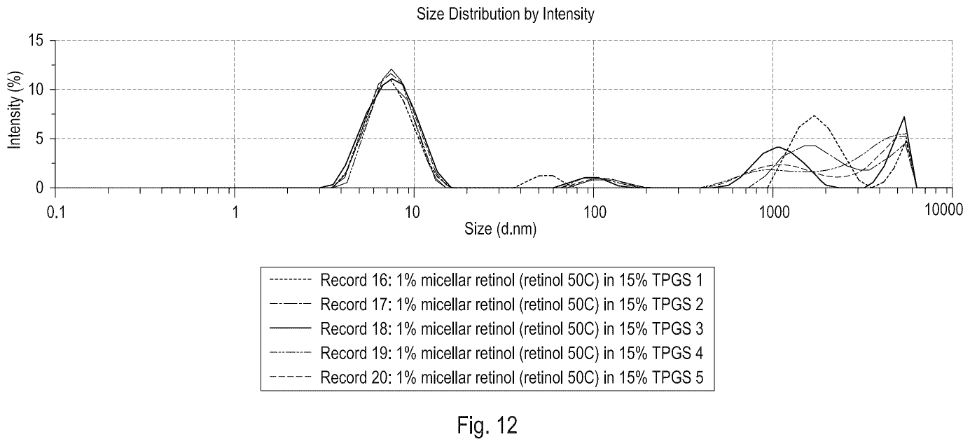

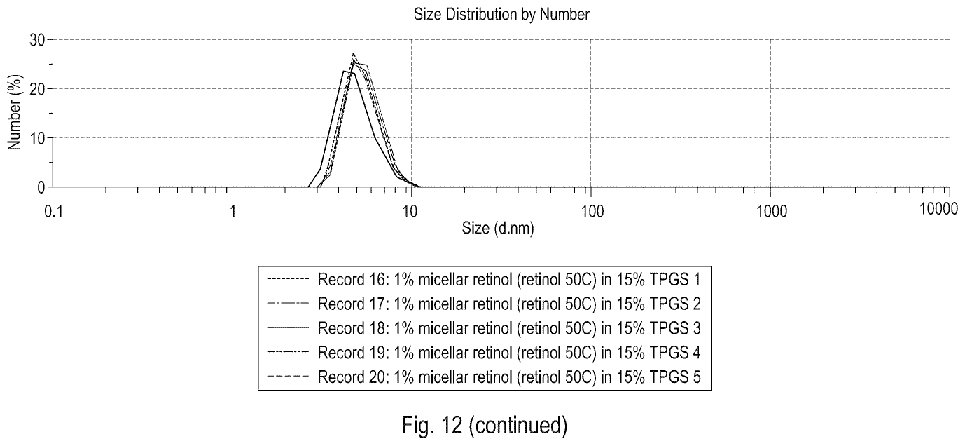

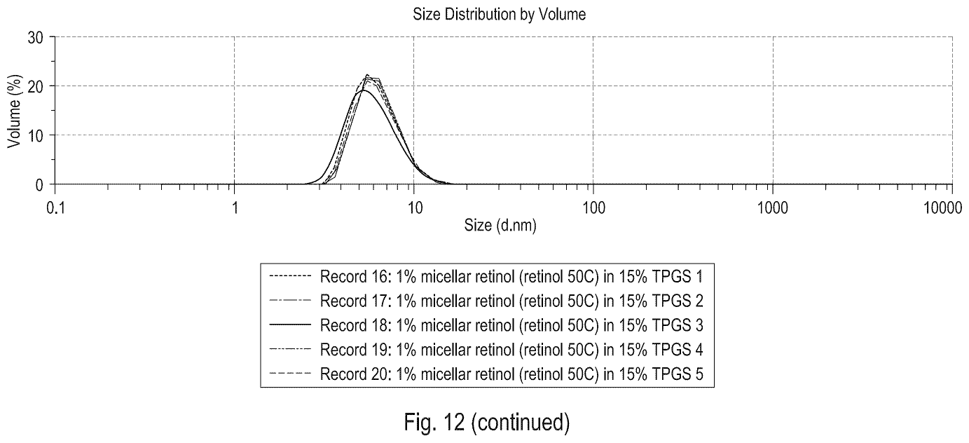

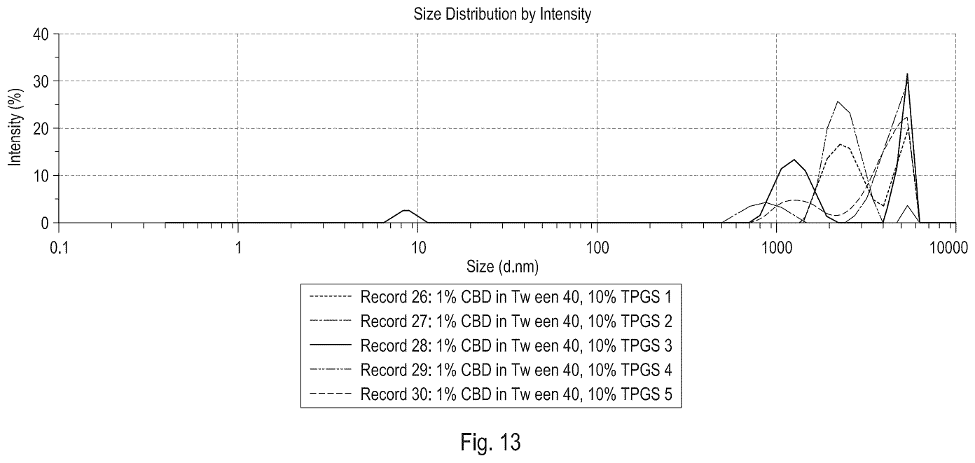

Micelles can be successfully made containing 1% retinol in polysorbate as solvent and with TPGS increased to 15% TPGS. The micelle size is between 5-15 nm (FIG. 12).

Micelles can be successfully made containing 10% TPGS 0.3% retinol in polysorbate.

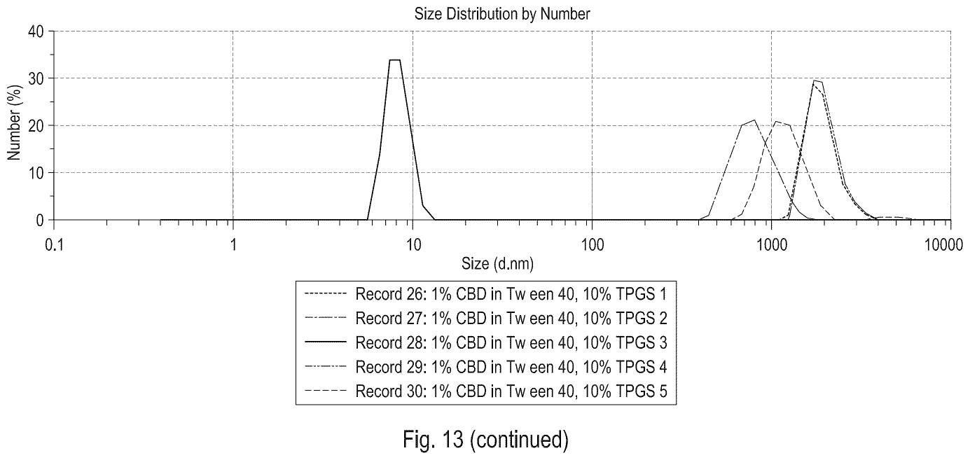

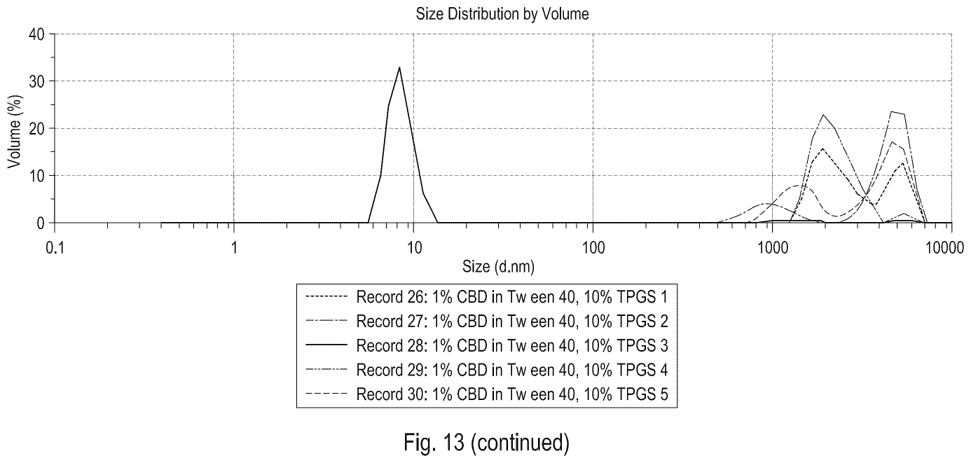

Micelles can be successfully made containing cannabidiol (CBD) in polysorbate. FIG. 13 shows that 1% CBD isolate in Polysorbate 40 (Tween® 40, 10% TPGS) using a solution of 10% CBD in Polysorbate 40. Results of 1% CBD in polysorbate shows a blend of large 600 nm and above and small micelles 8-15 nm, in similar numbers.

For comparison, the CBD results for ethanol are: 0.1 and 0.5% show clear micellar system, 5% is opaque. Size: 0.1% CBD between 8 and 35 nm with peak around 15-20 nm. 0.5% similar DLS results but some emerging data suggesting this approaches the CBD limit.

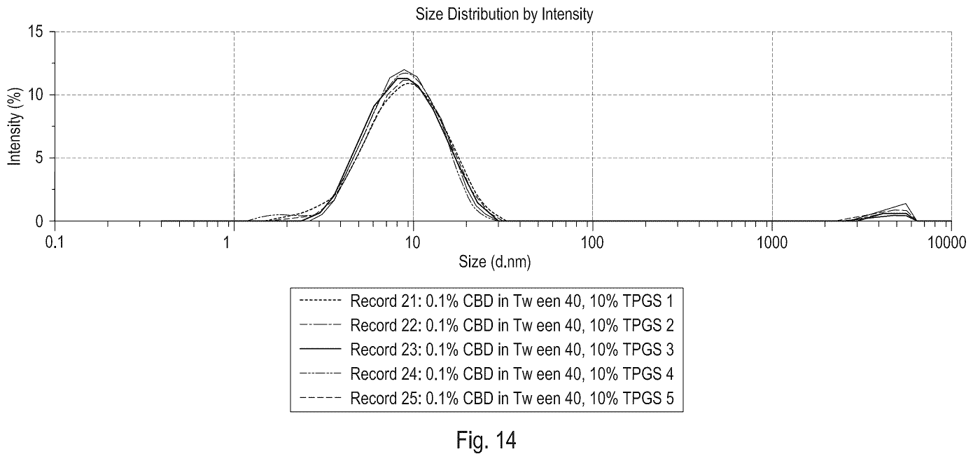

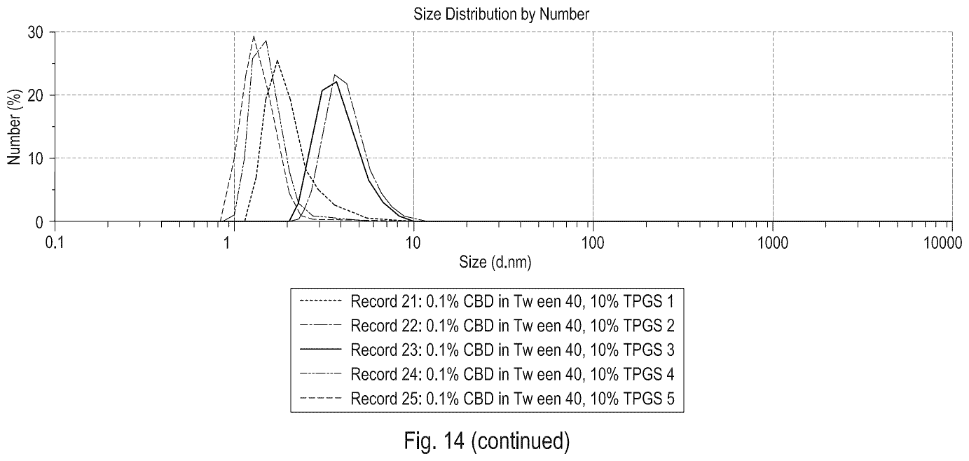

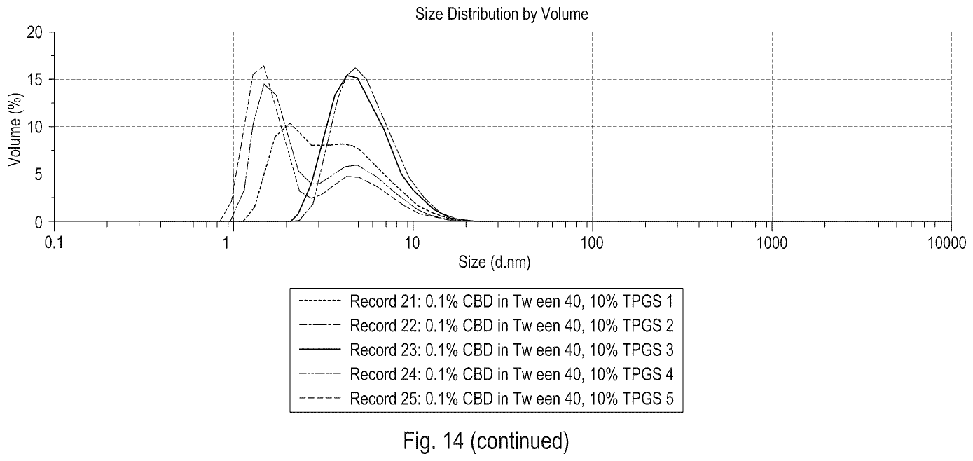

FIG. 14 shows DLS results of 0.1% CBD in polysorbate; micelles between 0.9-30 nm were seen with the majority between 1-15 nm.

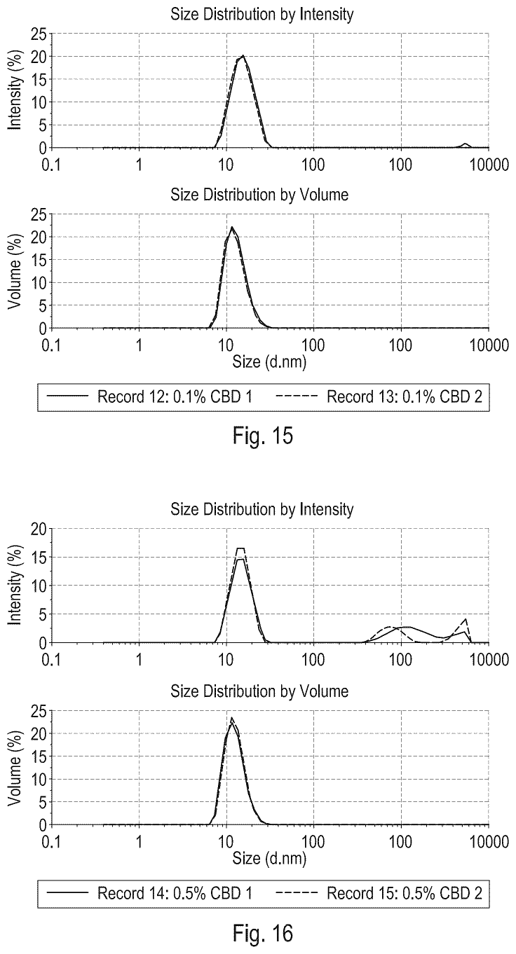

FIG. 15 shows DLS results for 0.1% CBD in ethanol with 10% TPGS d_{average}=16.26±0.7 nm.

FIG. 16 shows DLS results of 0.5% CBD in ethanol 20% TPGS d_{average}=28.92±7.40 nm.

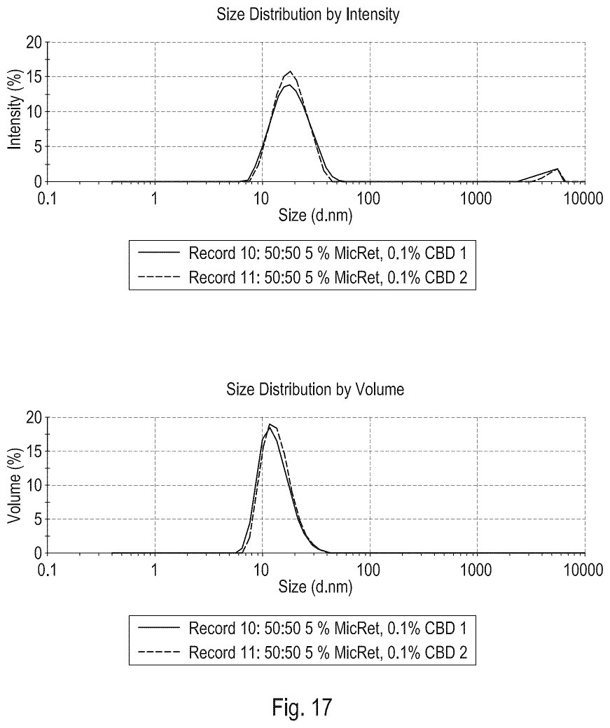

FIG. 17 shows DLS of mixed micelles of retinol and CBD for 5% retinol micelles mixed with 0.1% CBD micelles (final mix composed of a 2.5% retinol and 0.05% CBD) as a 50:50 blend. Micelles were observed of similar size to the premixed components, showing that diluting 5% retinol with 0.1% CBD micelles gives a clear micellar system of size between 3 and 35 nm. The peak size distribution by intensity or volume was around 10-20 nm. Blending can be adapted to give higher CBD and lower retinol i.e. if using a 3% retinol micellar stock use 10% of this and 90% of CBD micelle stock to give 0.3% retinol and 0.9% CBD, this approach of mixing micelles can be used for curcumin too. Therefore, it is possible to mix micelles.

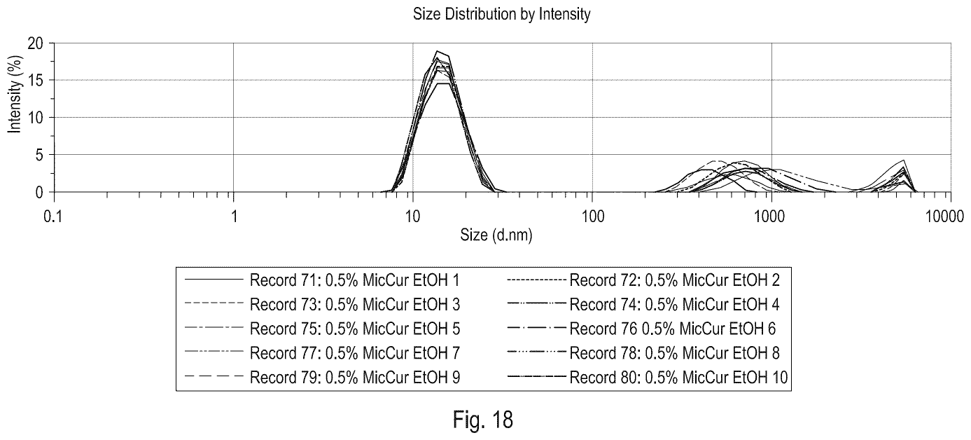

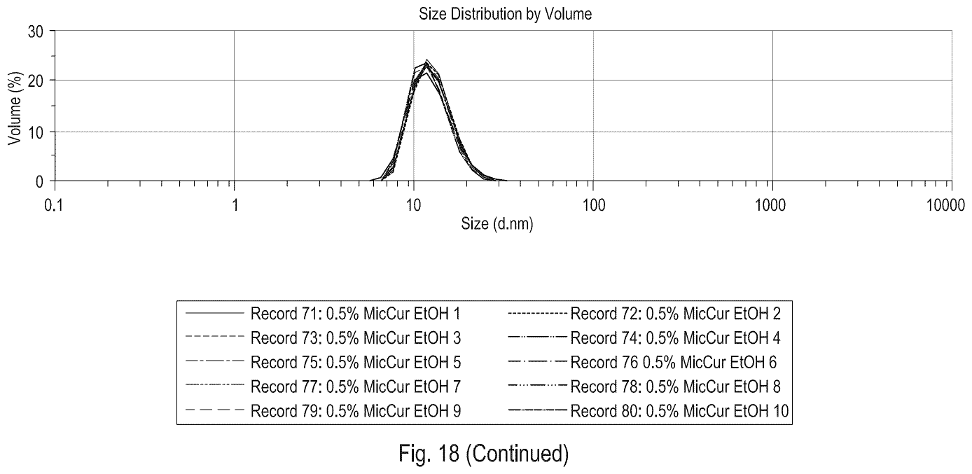

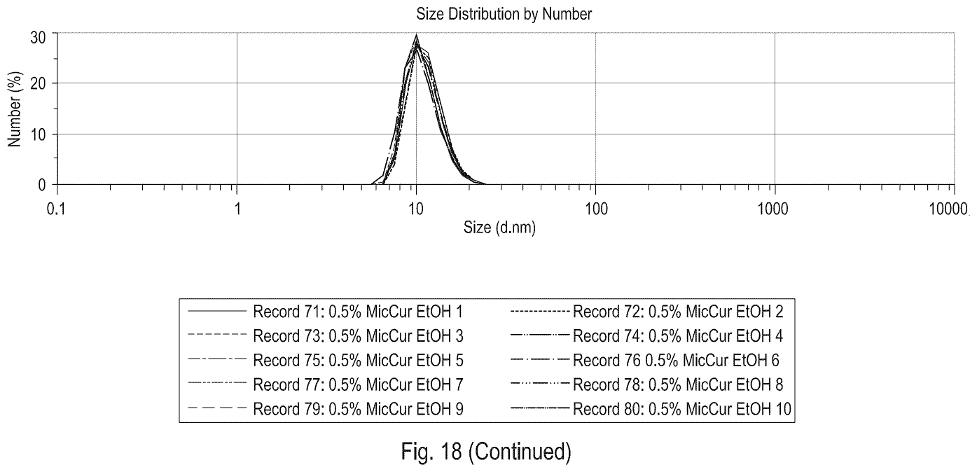

FIG. 18 shows DLS results of 0.5% micellar curcumin in ethanol, with peak (average) size (diameter) distribution by intensity or volume of around 10-20 nm.

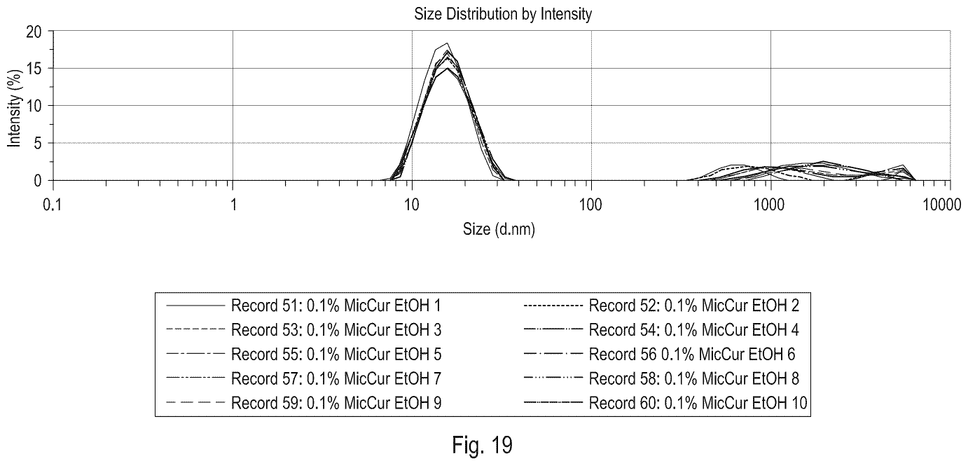

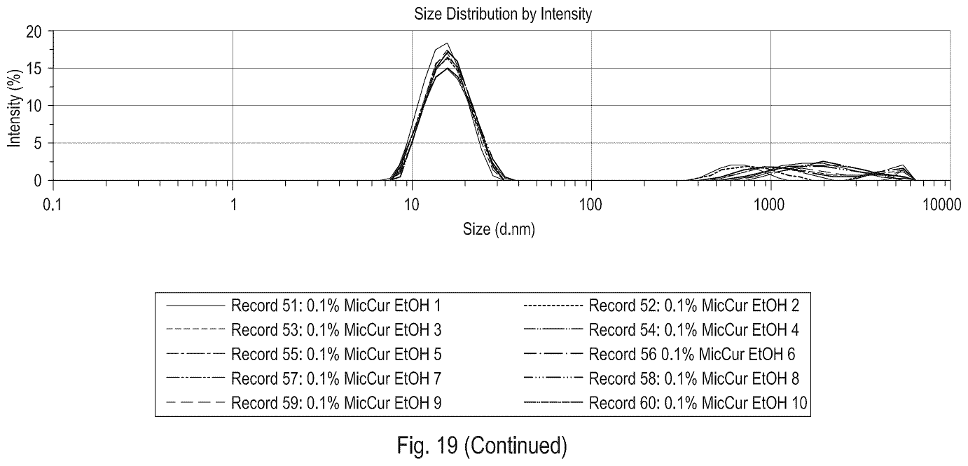

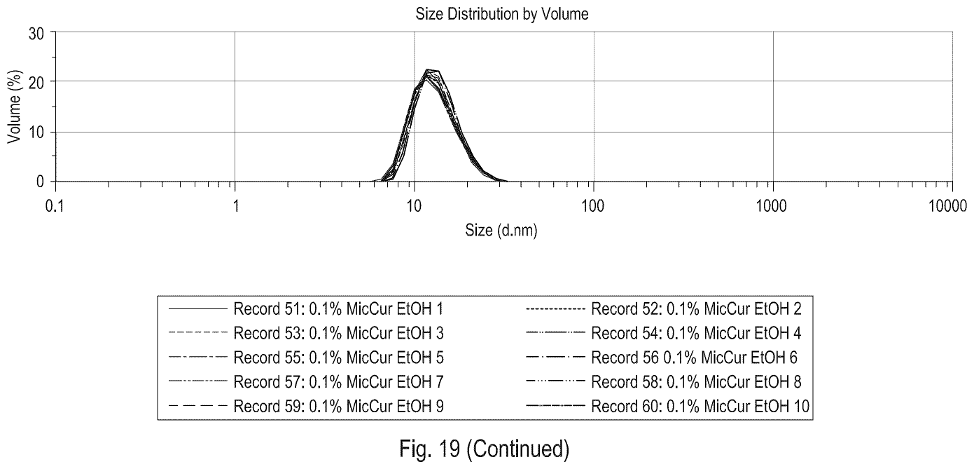

FIG. 19 shows DLS results of 0.1% micellar curcumin in ethanol, with peak (average) size (diameter) distribution by intensity or volume of around 10-20 nm.

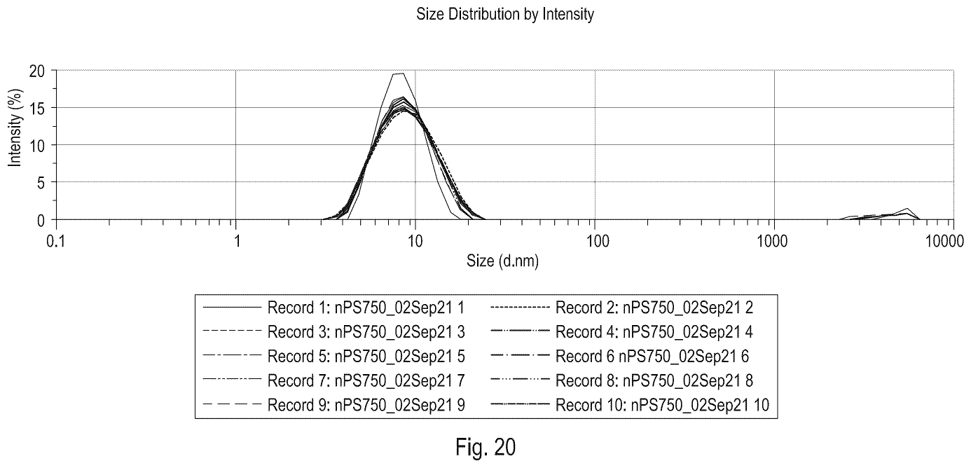

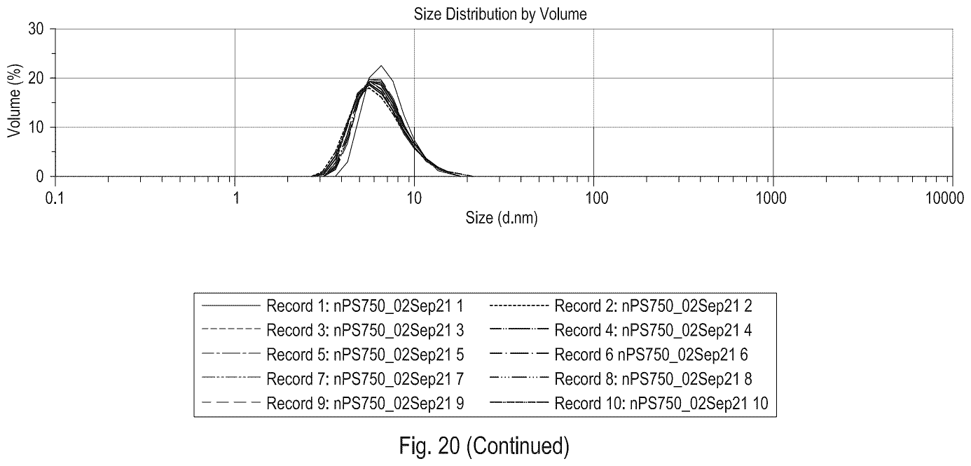

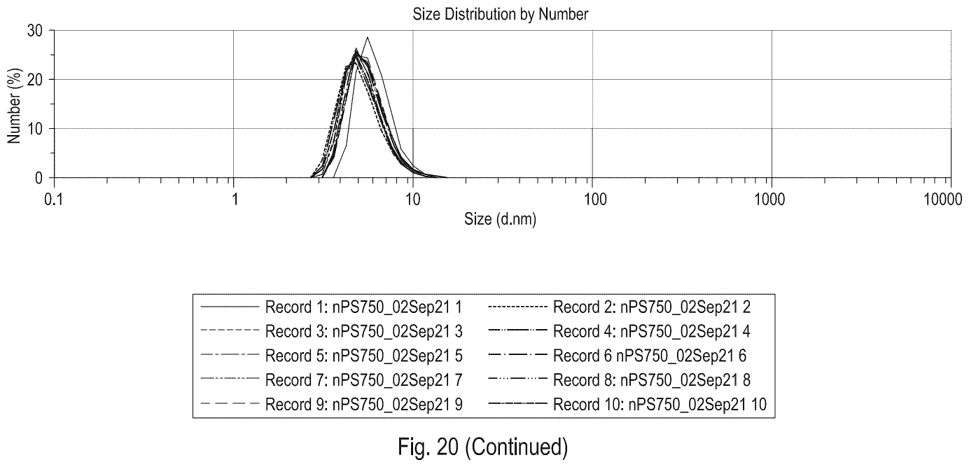

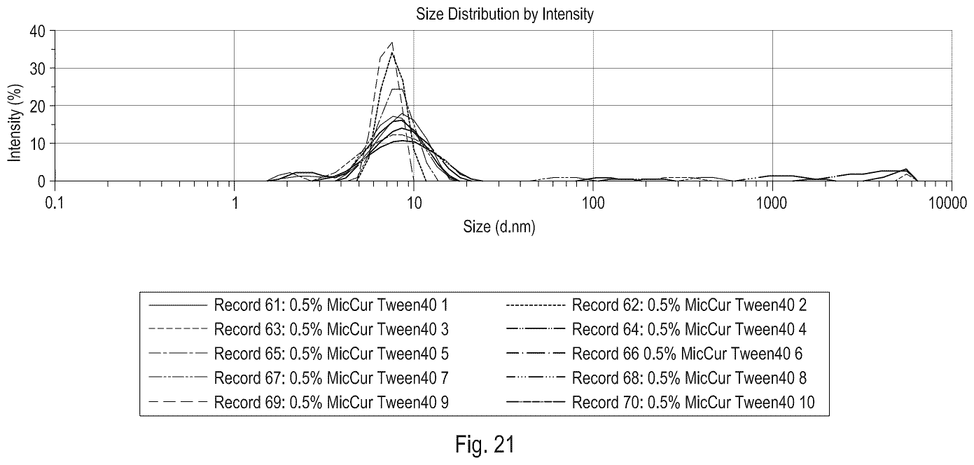

FIG. 20 shows DLS results of other micellar compositions comprising curcumin, having a micellar range of 2-20 nm (by intensity, volume or number), and a peak (average size distribution of about 5-8 nm (by intensity, volume or number).

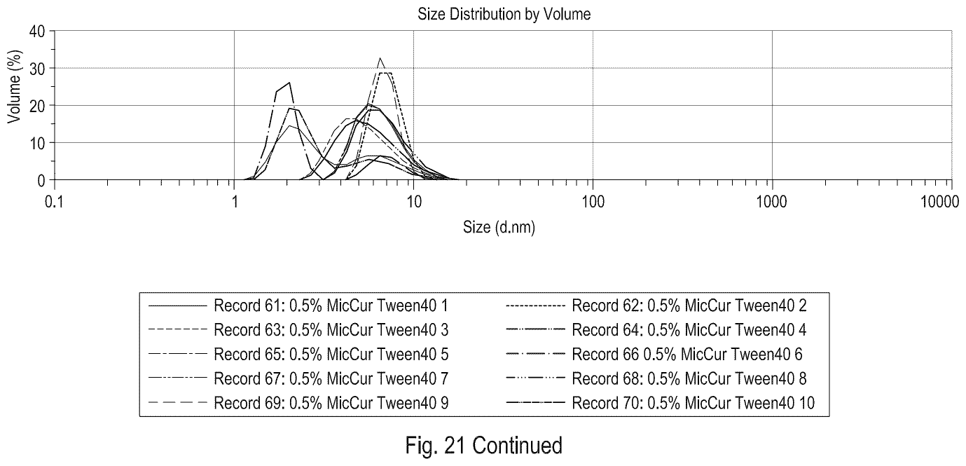

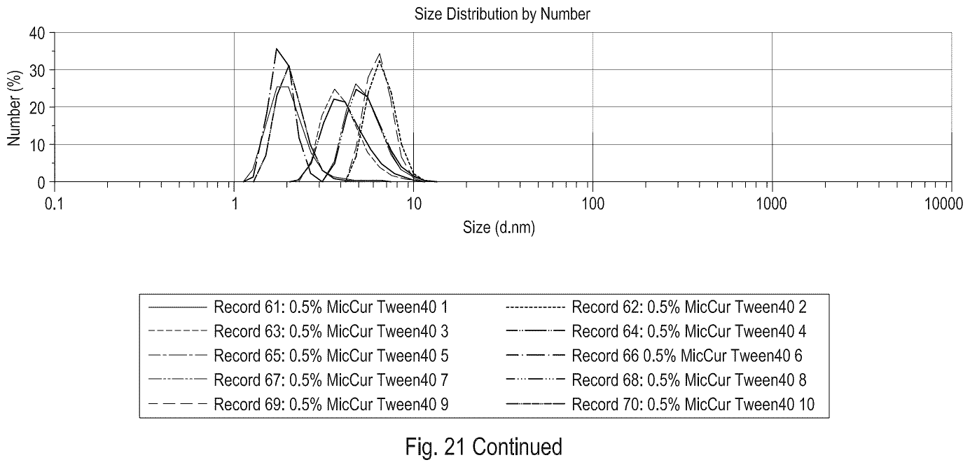

FIG. 21 shows DLS results of 0.5% curcumin micelles in Tween® 40, having a micellar range of 1-20 nm (by intensity, volume or number), and a peak (average size distribution of about 1-10 nm (by intensity, volume or number).

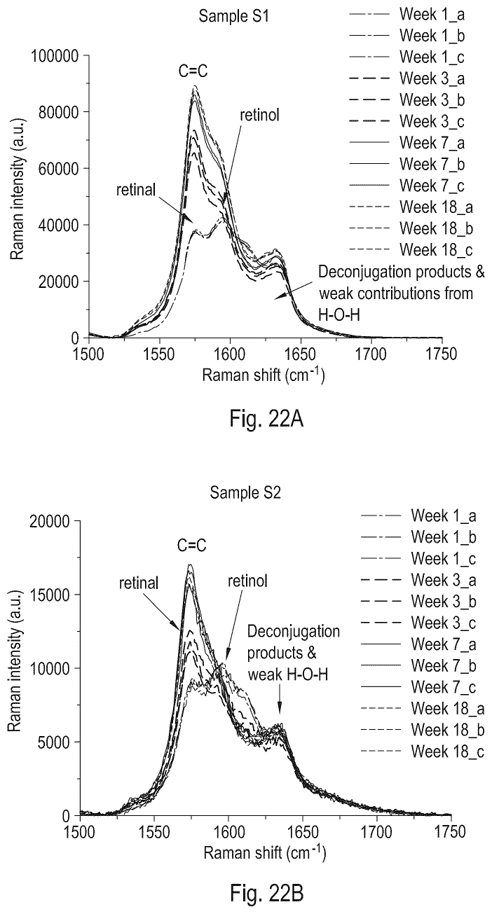

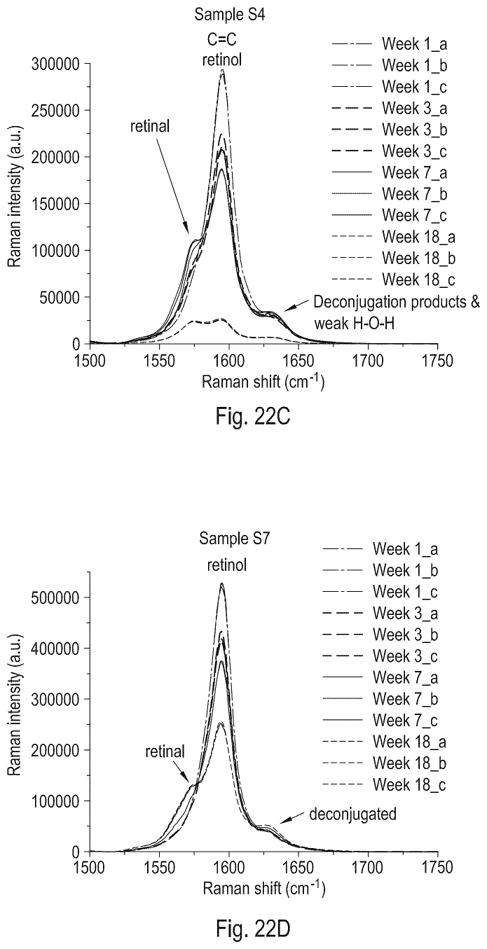

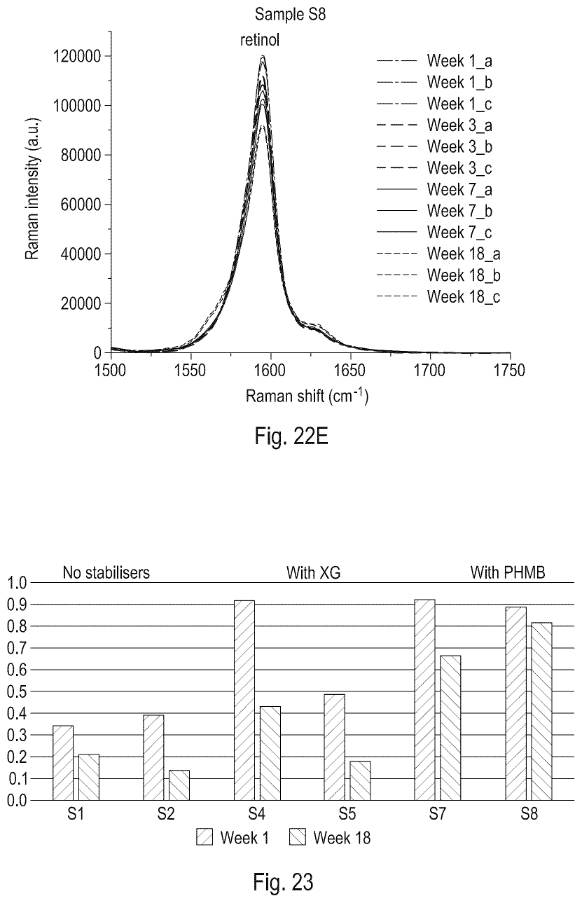

FIG. 22 shows the results of a stability study performed using Raman spectroscopy. The spectral region of 1600-1700 cm⁻¹ primarily consists of signal contributions from retinoid deconjugation products, but also some broad low intensity H—O—H signal contributions from water (centred at ˜1650 cm⁻¹). The spectral interpretation was guided by information in the reference N. Failloux et al., Applied Spectroscopy, 2003, 57:9, 1117-1122.

FIG. 22A shows the results for sample S1: 5% micellar retinol, no stabilisers. Over the duration of the study, the proportion of retinol decreased while retinal increased.

FIG. 22B shows the results for sample S2: 1% micellar retinol, no stabilisers. As for sample S1, over time, it appears that the proportion of retinol decreases while retinal increases.

FIG. 22C shows the results for sample S4: 5% micellar retinol with xanthan gum. Total Raman signal intensity falls with time, due to sample ageing. Compared to sample S1 (without xanthan gum), this sample had a much higher proportion of retinol. Retinal was barely detectable until week 3-7. By week 18 the ratio of retinol to retinal was approaching that seen during week 1 for sample S1.

FIG. 22D shows the results for sample S7: 5% micellar retinol with 0.1% PHMB. Total Raman signal intensity falls with time, due to sample ageing. Compared to sample S1 (without PHMB), this sample had a much higher proportion of retinol. Retinal was barely detectable until week 7-18. By week 18 the ratio of retinol to retinal was still greater than that seen during week 1 for sample S1. A small proportion of deconjugated products were also detected.

FIG. 22E shows the results for sample S8: 1% micellar retinol with 0.1% PHMB. This sample, containing PHMB, had a much higher proportion of stabilised retinol. Retinal was barely detectable, even at week 18.

FIG. 23 shows calculated proportion of retinol, compared to total retinol and retinal.

$$Retinol content ( i . e . proportion ) = [ retinol ] / [ retinol + retinal ]$$

The ratios were calculated using the peak areas of the retinol and retinal component fits.

The results are consistent with retinol stabilisation in the presence of xanthan gum and/or PHMB, with the formulation containing PHMB exhibiting the most improved performance.

This indicates that PHMB and/or xanthan gum can be used to tailor the amounts of retinoid (e.g. retinol and/or retinal) in the formulation. There is a reversible reaction from retinol to retinal and within biological processes retinol transforms to retinal which when attached to receptors in tissue expresses retinoic acid which then binds to retinoic acid receptors to activate generative and regenerative cellular processes through gene expression.

Retinal within the formulation can be beneficial to these processes if delivered into tissue, retaining retinol in the formulation at higher levels can be beneficial for controlling or slowing biological processes to control the generative and regenerative mechanisms of action.

Comparative Example: The Encapsulation of Retinol (0.3%) in Soy Bean Oil and Caprylic Triglyceride Solvents was Attempted. However, No Micelles Under 50 nm were Seen. Most particles observed were over 100 nm. The solution was not clear, suggesting the formation of large (over 100 nm) micelles.

Our successful micellar solutions are transparent with slight yellowing due to retinol (as with curcumin) and clear transparent for CBD.

Further studies on skin model penetration were conducted with 0.3% retinol, with and without xanthan gum, and compared with a leading clinical formulation.

| | | | | Weight of |
|---|---|---|---|---|
| | | | Weight of | retinol applied |
| | | Weight of | retinol | on the skin (mg) |
| | | formulation | applied on | based on the |
| Sample | Sample | applied on | the skin | diffusion area |
| ID | Description | the skin (g) | (mg)* | (mg/cm²) |
| S1 | 0.3% Micellar | 0.167 | 0.5 | 0.5 (1.00 cm²) |
| | Retinol solution | | | |
| | diluted in H₂O | | | |
| S3 | 0.3% Micellar | 0.300 | 0.9 | 0.5 (1.78 cm²) |
| | Retinol solution | | | |
| | diluted in 0.3% | | | |
| | w/v xanthan gum | | | |
| | (XG) | | | |

*assuming that all formulations contain 0.3% w/w of retinol

### Results

A dose of retinol of 500 μg/cm² was applied to the membrane. The amount of retinol recovered was determined.

| | Retinol Recovery (μg retinol/cm²) | | |
|---|---|---|---|
| Sample | Receptor Fluid | | Membrane surface |
| ID | after 24 h | Strat-M membrane | (has not penetrated) |
| S1 | ND | 20.53 | ND (not detected) |
| S3 | ND | 15.29 | ND |

The results indicate that retinol was not detected in the receptor fluid or on the membrane surface indicating the majority of the retinol has entered the membrane but not passed through indicating the delivery of retinol is not systemic. This is important as retinol (i.e. vitamin A) toxicity concerns limit the dermal delivery concentration of retinoids.

Therefore, the compositions of the invention overcome toxicity concerns with the delivery of high concentrations of retinol.

The use of xanthan gum as a thickener and stabiliser does not unduly impact on the delivery of retinol over 24 hours. However, the use of xanthan gum may beneficially slow the delivery of the retinol to deliver a more controlled dose. The skilled person will understand that the rate of dosage can be tailored through adjusting viscosity with thickeners such as xanthan gum.

### Effect of Retinoid Concentration on Particle Size in Aqueous Micellar Retinoid

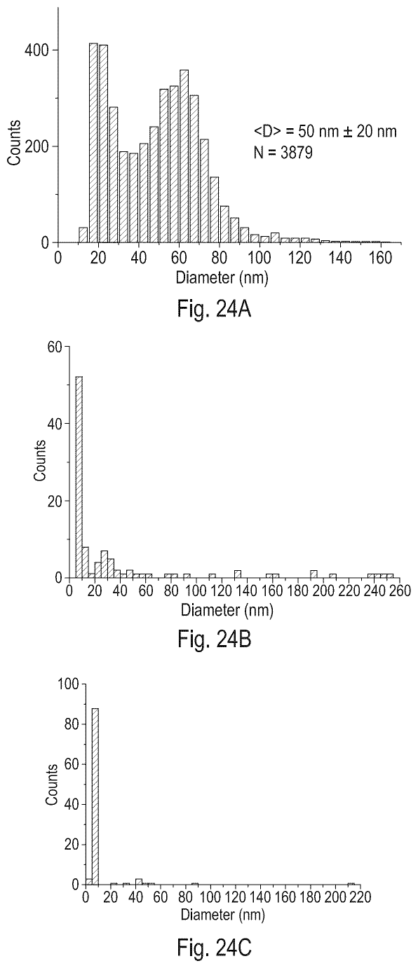

FIG. 24 shows that dilution brings more consistent micelles.

FIG. 24A shows the particle size distribution for a sample containing 5% retinol, where the particle size range is mostly from 15 to 80 nm, and very few particles are above 100 nm.

FIG. 24B shows the particle size distribution for a sample containing 1% retinol, where the particle size range is mostly from 5 to 15 nm, and very few particles are above 40 nm.

FIG. 24C shows the particle size distribution for a sample containing 0.3% retinol, where the particle size range is mostly from 5 to 10 nm, and very few particles are above 10 nm.

The present invention is not limited to 5% retinol. Other formulations containing different concentrations of retinol, or other retinoids, could be made (at different retinol %). These may be more stable and contain consistent micelles to produce a stable ‘stock’ of micelles.

1. The method of claim 2, wherein the method of cosmetic treatment comprises topical application of a composition to the subject's skin

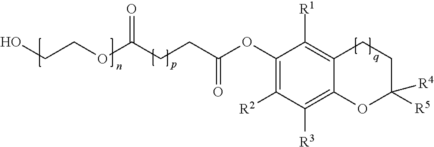

2. A method of treatment comprising topical administration of a composition to a subject, wherein the composition comprises micelles, the micelles comprising an aqueous outer phase that encapsulates an inner phase, wherein the micelles have a diameter of 100 nm or less, and wherein the aqueous outer phase comprises a compound of Formula (I), or a composition thereof 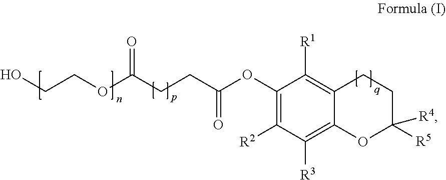
wherein:
R¹, R², R³ and R⁴ are each independently H or a C1-6 hydrocarbon group that is optionally substituted by one or more Y group;
R⁵ is a C6-30 hydrocarbon group;
n is from 3 to 100;
p is from 0 to 6;
q is from 0 to 3; and
Y is selected from the list consisting of ether, ester, sulfone, sulfoxide, amide, amine e.g. secondary amine and tertiary amine), boronate ester, ketone and aldehyde.

3. The method of claim 2, wherein the composition comprises a biologically active agent.

4. The method of claim 3, wherein the composition includes the biologically active agent in a concentration of is from 1_ng/ml to 10 μg/mL.

5. (canceled)

6. The method claim 5, wherein the application or administration of the composition provides controlled release of the biologically active compound.

7-11. (canceled)

12. A composition comprising micelles, the micelles comprising an aqueous outer phase that encapsulates an inner phase, wherein the micelles have a diameter of 100 nm or less, and wherein the aqueous outer phase comprises a compound of Formula (I), or a composition thereof 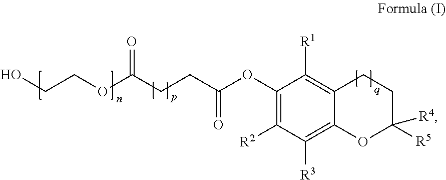
wherein:
R¹, R², R³ and R⁴ are each independently H or a C1-6 hydrocarbon group that is optionally substituted by one or more Y group;
R⁵ is a C6-30 hydrocarbon group;
n is from 3 to 100;
p is from 0 to 6;
q is from 0 to 3; and
Y is selected from the list consisting of ether, ester, sulfone, sulfoxide, amide, amine e.g. secondary amine and tertiary amine), boronate ester, ketone and aldehyde.

13. The composition of claim 12, wherein the composition is a medicament.

14. (canceled)

15. The method of claim 3, wherein the biologically active compound is a retinoid, cannabidiol (CBD), and/or curcumin.

16. The method of claim 15, wherein the retinoid is retinol.

17. The method of claim 15 or claim 16, wherein the composition contains the biologically active agent in an amount of 0.001 wt % or more, wherein the composition contains, water, ethanol and/or polysorbate, wherein the micelle size is from 5 to 50 nm, and wherein the amount of the compound of Formula (I) is from 1 wt % to 30 wt %.

18. The method of claim 16, wherein the inner phase comprises retinol in an amount of 0.0001 to 3w/w % retinol.

19. The method of claim 16, wherein the inner phase comprises CBD in an amount of 0.00001 to 2w/w %.

20. The method of claim 16, wherein the inner phase comprises curcumin in an amount of 0.0001 to 1w/w %.

21. The method of claim 2, wherein the aqueous outer phase additionally comprises polysorbate.

22. The method of claim 2, wherein the composition comprises a thickener and/or a stabiliser.

23. The method of claim 22, wherein the thickener and/or stabiliser is xanthan gum and/or polyhexamethylene biguanide (PHMB).

24. (canceled)

25. The method of claim 15, wherein the biologically active agent is a retinoid, curcumin and/or CBD, wherein the biologically active agent is present in an amount of 0.001 wt % or more, wherein the composition contains water, ethanol and/or polysorbate, wherein the micelle size is from 5 to 50 nm, and wherein the amount of the compound of Formula (I) is from 0.0001 wt % to 30 wt %.

26. A method for the preparation of the composition of claim 1, the method comprising:
providing a hydrophobic phase comprising an organic solvent;
adding the hydrophobic phase into an aqueous dispersion of the compound of Formula (I); and
optionally removing at least a portion of the organic solvent.

27. The method of claim 2, wherein the method is for scar recovery or wound care.
